(19) United States
(12) Patent Application Publication
(10) Pub. No.: US 2012/0225896 A1
(43) Pub. Date: Sep. 6, 2012

(54) NOVEL 2-PYRIDONE DERIVATIVE AND PHARMACEUTICAL PRODUCT CONTAINING SAME

(75) Inventors: Toru Miura, Higashimurayama-shi (JP); Junya Tagashira, Higashimurayama-shi (JP); Ryohei Sekimoto, Higashimurayama-shi (JP); Rie Ishida, Higashimurayama-shi (JP); Hitomi Aoki, Higashimurayama-shi (JP); Tadaaki Ohgiya, Higashimurayama-shi (JP)

(73) Assignee: KOWA COMPANY, LTD., Nagoya-shi, Aichi (JP)

(21) Appl. No.: 13/509,130

(22) PCT Filed: Dec. 22, 2010
(86) PCT No.: PCT/JP2010/007417

§ 371 Date: May 10, 2012

(30) Foreign Application Priority Data
Dec. 22, 2009 (JP) 2009-290149

(51) Int. Cl. A61K 31/506; A61K 31/4439; C07D 413/14; C07D 401/14; C07D 401/10; A61P 3/10; A61P 9/12; A61P 9/00; A61P 13/12; A61P 9/10; A61P 3/00; A61K 31/444; C07D 413/10

(52) U.S. Cl. 514/273; 514/333; 514/340; 544/298; 546/256; 546/268.4; 546/269.1

(57) ABSTRACT

Disclosed is a novel 2-pyridone derivative represented by general formula (1), which has both angiotensin II receptor antagonist activity and PPAR-γ activating activity and is useful as a prophylactic and/or therapeutic agent for cardiovascular diseases and metabolic diseases. Also disclosed is a pharmaceutical composition which contains the novel 2-pyridone derivative. In general formula (1), R¹ represents a C₁₋₆ alkyl group or a C₁₋₆ alkoxy group; R² represents a C₁₋₆ alkyl group or a C₃₋₈ cycloalkyl group; R³ represents a C₁₋₆ alkyl group, a C₆₋₁₀ aryl-C₁₋₆ alkyl group, a C₁₋₆ alkoxy-C₁₋₆ alkyl group or a group represented by formula (2) (wherein A represents a nitrogen atom or CH, and R⁵ represents a hydrogen atom or a C₁₋₆ alkoxy group); and R⁴ represents a group represented by formula (3) or (4).

### TECHNICAL FIELD

The present invention relates to a novel 2-pyridone derivative that has both angiotensin II antagonistic activity and PPARγ activation activity, and a pharmaceutical agent containing the same.

### BACKGROUND ART

In recent years, disorders like diabetes, hypertension, dyslipidemia and obesity which can be a risk factor for arteriosclerotic disorders have been rapidly increasing due to changes in life style with improvements in living standard, i.e., high calorie and high cholesterol type diet, obesity, lack of exercise, aging, and the like. It is known that, although being a risk factor independent of each other, overlap of the disorders can cause an occurrence of arteriosclerotic disorders at higher frequency or aggravation of the disorders. As such, with the understanding of a condition having a plurality of risk factors for arteriosclerotic disorders as metabolic syndrome, efforts have been made to elucidate the cause of the syndrome and to develop a therapeutic method therefor.

Angiotensin II (herein below, it may be also abbreviated as AII) is a peptide that is found to be an intrinsic pressor substance produced by renin-angiotensin system (i.e., RA system). It is believed that pharmacological inhibition of angiotensin II activity can lead to treatment or prevention of circulatory disorders like hypertension. Accordingly, an inhibitor for angiotensin converting enzyme (ACE) which inhibits the enzyme promoting the conversion of angiotensin I (AI) to angiotensin II has been clinically used as an inhibitory agent for RA system. Furthermore, an orally administrable AII receptor blocker (Angiotensin Receptor Blocker: ARB) has been developed, and losartan, candesartan, telmisartan, valsartan, olmesartan, and irbesartan, and the like are already clinically used as a hypotensive agent. It is reported by many clinical or basic studies that, as having not only a hypotensive activity but also other various activities including an anti-inflammatory activity, an endothelial function improving activity, a cardiovascular remodeling inhibiting activity, an oxidation stress inhibiting activity, a proliferation factor inhibiting activity, and insulin resistance improving activity, and the like, ARB is useful for cardiovascular disorders, renal diseases, and arteriosclerosis, and the like (Non-Patent Documents 1 and 2). Most recently, it is also reported that ARB particularly has a kidney protecting activity which does not depend on a hypotensive activity (Non-Patent Document 3).

Meanwhile, three isoforms, i.e., α, γ, and δ, have been identified so far as peroxisome proliferator-activated receptors (PPARs) which belong to a nuclear receptor superfamily. Among them, PPARγ is an isoform that is most abundantly expressed in an adipose tissue and it plays an important role in differentiation of adipocytes or metabolism of glycolipids. Currently, thiazolidinedione derivatives (i.e., TZD) like pioglitazone or rosiglitazone are clinically used as a therapeutic agent for diabetes having PPARγ activation activity, and they are known to have an activity of improving insulin resistance, glucose tolerance, and lipid metabolism, and the like. Further, it is recently reported that, based on activation of PPARγ, TZD exhibits various activities including a hypotensive activity, an anti-inflammatory activity, an endothelial function improving activity, a proliferation factor inhibiting activity, and an activity of interfering RA system, and the like. It is also reported that, according to such multiple activities, TZD shows a kidney protecting activity particularly in diabetic nephropathy without depending on blood sugar control (Non-Patent Documents 4, 5, 6, 7, and 8). Meanwhile, there is also a concern regarding adverse effects of TZD caused by PPARγ activation like body fluid accumulation, body weight gain, peripheral edema, and pulmonary edema (Non-Patent Documents 9 and 10).

It has been recently reported that telmisartan has a PPARγ activation activity (Non-Patent Document 11). It has been also reported that the irbesartan has the same activity (Non-Patent Document 12). These compounds have both a RA system inhibiting activity and a PPARγ activation activity, and thus are expected to be used as an integrated agent for prevention and/or treatment of circulatory disorders (e.g., hypertension, heart diseases, angina pectoris, cerebrovascular disorders, cerebral circulatory disorders, ischemic peripheral circulatory disorders, and renal diseases, and the like) or diabetes-related disorders (e.g., Type II diabetes, diabetic complications, insulin resistant syndrome, metabolic syndrome, hyperinsulinemia, and the like) without increasing a risk of body fluid accumulation, body weight gain, peripheral edema, pulmonary edema, or congestive heart failure that are concerned over the use of TZD (Patent Document 1). Among them, for diabetic nephropathy, a synergistic prophylactic and/or therapeutic effect is expected from multiple kidney protecting activity based on activities of RA system inhibition and PPARγ activation.

As compounds having such activities, pyrimidine and triazine derivatives (Patent Document 1), imidazopyridine derivatives (Patent Document 2), indole derivatives (Patent Document 3), imidazole derivatives (Patent Document 4), and condensed ring derivatives (Patent Document 5) have been reported.

Among them, Patent Document 1 discloses compounds represented by the following formula (A):

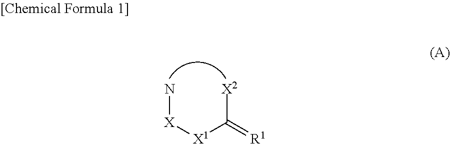

[wherein, R¹ represents an oxygen atom, a sulfur atom, or the like,

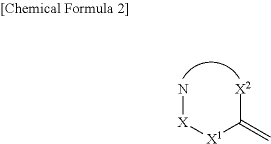

the above moiety represents a group represented by the following formula (Aa) to (Ac):

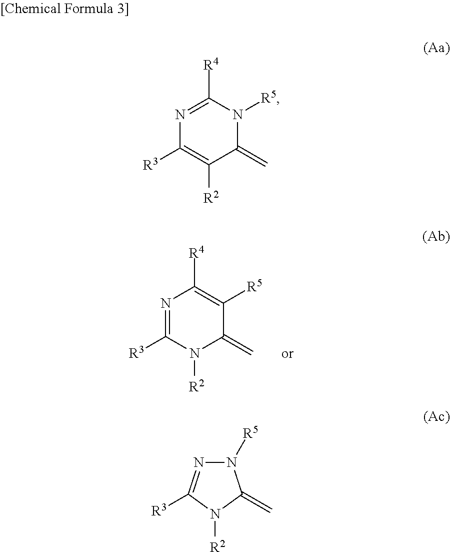

R² represents a group represented by the following formula (Ad):

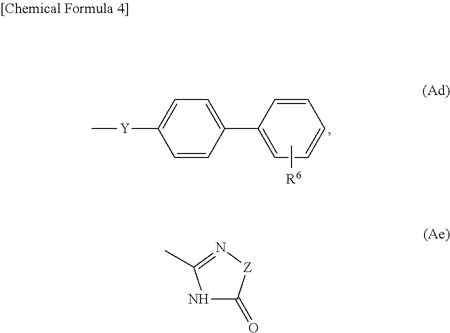

R⁶ represents a group represented by the formula (Ae) above, Z represents an oxygen atom or S(O)ₙ, n represents an integer of from 0 to 2, Y represents a C₁-C₄ alkylene group or the like, R³ and R⁴ each independently represent a group selected from a hydrogen atom, a C₁-C₆ alkyl group, a C₃-C₆ cycloalkyl group, a C₁-C₆ alkoxy group, and a C₁-C₆ alkylamino group, and R⁵ represents a group selected from a hydrogen atom, a C₁-C₆ alkyl group, and a C₂-C₆ alkenyl group]. However, the heterocycle moiety of the literature represents the formula (Aa) to (Ac), which is different from the 2-pyridone skeleton of the invention.

Meanwhile, a Patent Document 6 discloses compounds represented by the following formula (B):

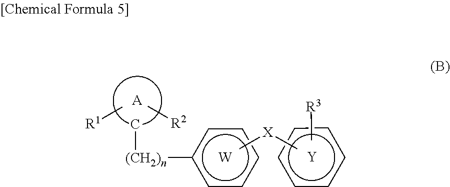

[wherein, ring A is an aromatic heterocyclic group in which the ring is composed of 5 to 10 ring-constituting atoms and may have a substituent groups other than R¹ and R², in which R¹ is a hydrocarbon residue that may be bonded through a hetero atom or may be substituted, R² is a group capable of releasing protons in vivo or another group convertible thereto, R³ is a 5- to 7-membered heterocyclic residue having a carbonyl group, a thiocarbonyl group, a sulfur atom which may be oxidized already or a ring-constituting group which may be transformed into such group as a ring-constituting group, in which the ring may be substituted, X represents that a ring Y is bonded directly or through a spacer of a chain with two or less atoms to a ring W, the ring W and the ring Y are each an aromatic hydrocarbon or an aromatic heterocyclic residue that may be substituted, and n represents an integer of from 1 to 3].

However, it is not described or suggested that the compound of the literature has a PPARγ activation activity as a pharmacological activity or has a therapeutic effect on diabetes, obesity, or metabolic syndrome. Further, from the viewpoint that it characteristically has a group capable of releasing protons such as a carboxyl group, a tetrazolyl group, a trifluoromethane sulfonic acid amide group, a phosphoric acid group, a sulfonic acid group, and the like as R², the compound of the literature is different from the compound of the invention.

Further, Patent Document 7 discloses compounds represented by the following formula (C):

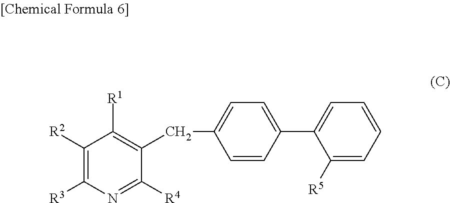

[in the formula, R¹, R², R³, and R⁴ each independently represent a hydrogen, a lower alkyl group, a lower alkenyl group, a hydroxymethyl group, a hydroxy group, a carboxymethyl group, a nitro group, a methoxy group, a sulfonylamino group, or halogen, and R⁵ represents a carboxy group or a tetrazolyl group].

However, it is not described or suggested that the compound of the literature has a PPARγ activation activity as a pharmacological activity or has a therapeutic effect on diabetes, obesity, or metabolic syndrome. Further, from the viewpoint that no substituent group is present on the nitrogen atom of the pyridine ring, it is different from the compound of the invention.

### PRIOR ART DOCUMENT

### Patent Document

Patent Document 1: WO 2008/062905
Patent Document 2: WO 2008/084303
Patent Document 3: WO 2008/096820
Patent Document 4: WO 2008/096829
Patent Document 5: WO 2008/143262
Patent Document 6: Japanese Patent Application Laid-Open (JP-A) No. 07-070118
Patent Document 7: Japanese Patent Application Laid-Open (JP-A) No. 05-310696

### Non-Patent Document

Non-Patent Document 1: Amer. J. Hypertension, 18, 720 (2005)
Non-Patent Document 2: Current Hypertension Report, 10, 261 (2008)
Non-Patent Document 3: Diabetes Care, 30, 1581 (2007)
Non-Patent Document 4: Kidney Int., 70, 1223 (2006)
Non-Patent Document 5: Circulation, 108, 2941 (2003)
Non-Patent Document 6: Best Pract. Res. Clin. Endocrinol. Metab., 21(4), 687 (2007)
Non-Patent Document 7: Diab. Vasc. Dis. Res., 1(2), 76 (2004)
Non-Patent Document 8: Diab. Vasc. Dis. Res., 2(2), 61 (2005)
Non-Patent Document 9: J. Clin. Invest., 116(3), 581 (2006)
Non-Patent Document 10: FASEB J., 20(8), 1203 (2006)
Non-Patent Document 11: Hypertension, 43, 993 (2004)
Non-Patent Document 12: Circulation, 109, 2054 (2004)

### SUMMARY OF THE INVENTION

### Problems to be Solved by the Invention

An object of the present invention is to provide a novel compound that is useful as a pharmaceutical agent for preventing and/or treating hypertension as a circulatory disorder, diabetes as a metabolic disease, or the like, and a pharmaceutical composition using the same.

### Means for Solving the Problems

As a result of intensive studies to achieve the purpose described above, the inventors of the invention found that 2-pyridone derivative represented by the formula (1) is a compound that has excellent angiotensin II antagonistic activity and PPARγ activation activity, and therefore completed the invention.

Specifically, the present invention relates to the following inventions.

[1] A compound represented by the formula (1) below:

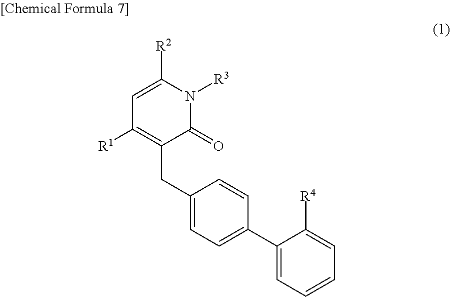

[in the formula, R¹ represents a C₁₋₆ alkyl group or a C₁₋₆ alkoxy group,

R² represents a C₁₋₆ alkyl group or a C₃₋₈ cyclolalkyl group,

R³ represents a C₁₋₆ alkyl group, a C₆₋₁₀ aryl-C₁₋₆ alkyl group, a C₁₋₆ alkoxy-C₁₋₆ alkyl group, or the following formula (2):

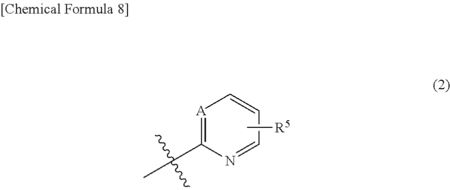

(in which A represents a nitrogen atom or CH and R⁵ represents a hydrogen atom or a C₁₋₆ alkoxy group), and

R⁴ represents the following formula (3) or the formula (4):

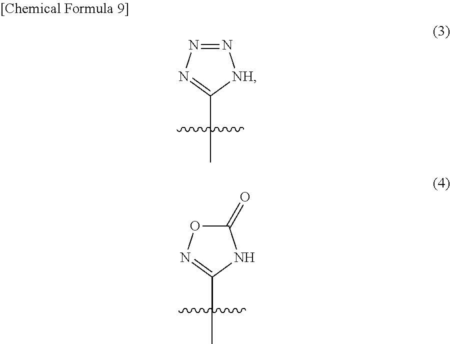

or a salt thereof, or a solvate thereof].

[2] The compound according to [1] or the salt thereof, or the solvate thereof, wherein the compound represented by the formula (1) is a compound selected from a group consisting of:
3-{[2′-(1H-tetrazol-5-yl)biphenyl-4-yl]methyl}-1-benz yl-4-butyl-6-methylpyridin-2(1H)-one,
3-{4′-[(1-benzyl-4-butyl-6-methyl-2-oxo-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-1-benzyl-6-methyl-4-propylpyridin-2(1H)-one,
3-{4′-[(1-benzyl-6-methyl-2-oxo-4-propyl-1,2-dihydropyridin-3-yl)methyl]biphenyl-2-yl}-1,2,4-oxadiazol-5(4H)-one
3-{[2′-(1H-tetrazol-5-yl)biphenyl-4-yl]methyl}-1-benzyl-6-methyl-4-pentylpyridin-2(1H)-one,
3-{4′-[(1-benzyl-6-methyl-2-oxo-4-pentyl-1,2-dihydropyridin-3-yl)methyl]biphenyl-2-yl}-1,2,4-oxadiazol-5(4H)-one
3-{[2′-(1H-tetrazol-5-yl)biphenyl-4-yl]methyl}-4-butyl-6-methyl-1-phenethylpyridin-2(1H)-one,
3-{4′-[(4-butyl-6-methyl-2-oxo-1-phenethyl-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-4-butyl-1-(2-methoxyethyl)-6-methylpyridin-2(1H)-one,
3-{4′-{[4-butyl-1-(2-methoxyethyl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-1-(5-ethoxypyrimidin-2-yl)-6-methylpyridin-2(1H)-one,
3-{4′-{[4-butyl-1-(5-ethoxypyrimidin-2-yl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-1-(5-methoxypyrimidin-2-yl)-6-methylpyridin-2(1H)-one,
3-{4′-{[4-butyl-1-(5-methoxypyrimidin-2-yl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-6-methyl-1-(pyridin-2-yl)pyridin-2(1H)-one,
3-{4′-{[4-butyl-6-methyl-2-oxo-1-(pyridin-2-yl)-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-1-benzyl-6-methyl-4-propoxypyridin-2(1H)-one, and
3-{4′-[(1-benzyl-6-methyl-2-oxo-4-propoxy-1,2-dihydro pyridin-3-yl)methyl]biphenyl-2-yl}-1,2,4-oxadiazol-5(4H)-one.

The alkyl group such as butyl in the nomenclature of the above-mentioned compounds represents a straight (normal) chain unless particularly designated.

[3] A pharmaceutical composition containing the compound or the salt thereof, or the solvate thereof described in the above [1] or [2], and a pharmaceutically acceptable carrier.

[4] A pharmaceutical composition containing the compound or the salt thereof, or the solvate thereof described in the above [1] or [2] as an effective component, having both angiotensin II receptor antagonistic activity and PPARγ activation activity.

[5] An agent for preventing and/or treating a circulatory disorder containing as an effective component the compound or the salt thereof, or the solvate thereof described in the above [1] or [2].

[6] The agent for preventing and/or treating a circulatory disease described in the above [5], wherein the circulatory disease is hypertension, heart disease, angina pectoris, cerebral vascular accident, cerebrovascular disorder, ischemic peripheral circulatory disorder, kidney disease, or arteriosclerosis.

[7] An agent for preventing and/or treating a metabolic disease containing as an effective component the compound or the salt thereof, or the solvate thereof described in the above [1] or [2].

[8] The agent for preventing and/or treating a metabolic disease described in the above [7], wherein the metabolic disease is Type II diabetes mellitus, diabetic complication (diabetic retinopathy, diabetic neuropathy, or diabetic nephropathy), insulin resistant syndrome, metabolic syndrome, or hyperinsulinemia.

[9] A method of preventing and/or treating a circulatory disease characterized in that an effective amount of the compound or the salt thereof, or the solvate thereof described in the above [1] or [2] is administered to a patient in need of the treatment.

[10] A method of preventing and/or treating a metabolic disease characterized in that an effective amount of the compound or the salt thereof, or the solvate thereof described in the above [1] or [2] is administered to a patient in need of the treatment.

[11] Use of the compound or the salt thereof, or the solvate thereof described in the above [1] or [2] for production of a preparation used for prevention and/or treatment of a circulatory disease.

[12] Use of the compound or the salt thereof, or the solvate thereof described in the above [1] or [2] for production of a preparation used for prevention and/or treatment of a metabolic disease.

[13] The compound or the salt thereof, or the solvate thereof described in the above [1] or [2] as a preventive and/or therapeutic agent having both an angiotensin II receptor antagonist activity and a PPARγ activation activity.

### Effects of the Invention

The 2-pyridone derivative represented by the formula (1) of the invention or a salt thereof, or a solvate thereof exhibits a potent antagonistic activity for an angiotensin II receptor, and can be appropriately used as an effective component for an agent for preventing and/or treating a disease related with angiotensin II, for example a circulatory disease such as hypertension, heart disease, angina pectoris, cerebral vascular accident, cerebrovascular disorder, ischemic peripheral circulatory disorder, kidney disease, and arteriosclerosis.

Further, the 2-pyridone derivative represented by the formula (1) of the invention or a salt thereof, or a solvate thereof has a PPARγ activation activity and can be appropriately used as an effective component for an agent for preventing and/or treating a disease related with PPARγ, for example metabolic disease such as arteriosclerosis, Type II diabetes mellitus, diabetic complication (diabetic retinopathy, diabetic neuropathy, or diabetic nephropathy), insulin resistance syndrome, syndrome X, metabolic syndrome, and hyperinsulinemia.

Still further, the 2-pyridone derivative represented by the formula (1) of the invention, or a salt thereof, or a solvate thereof has both an antagonistic activity for an angiotensin II receptor and PPARγ activation activity and can be appropriately used as an effective component for an agent for preventing and/or treating a disease related with both angiotensin II and PPARγ, for example, arteriosclerosis, diabetic nephropathy, insulin resistance syndrome, syndrome X, and metabolic syndrome.

### MODES FOR CARRYING OUT THE INVENTION

The “halogen atom” as used herein includes a fluorine atom, a chlorine atom, a bromine atom, an iodine atom, and the like.

The “C₁₋₆ alkyl group” and the “C₁₋₆ alkyl” as used herein mean a linear or branched hydrocarbon group having 1 to 6 carbon atoms, and examples thereof include a methyl group, an ethyl group, a n-propyl group, an isopropyl group, a n-butyl group, an isobutyl group, a t-butyl group, a n-pentyl group, a 2-methylbutyl group, a 2,2-dimethylpropyl group, a n-hexyl group, and the like.

The “C₁₋₆ alkoxy group” and “C₁₋₆ alkoxy” as used herein mean a linear or branched alkoxy group having 1 to 6 carbon atoms, and examples thereof include a methoxy group, an ethoxy group, a propoxy group, an isopropoxy group, a butoxy group, an isobutoxy group, a sec-butoxy group, a tert-butoxy group, a pentoxy group, an isopentoxy group, a neopentoxy group, a hexyloxy group, an isohexyloxy group, and the like.

The “C₃₋₈ cycloalkyl group” as used herein includes saturated or unsaturated monocyclic, polycyclic, or condensed cyclic cycloalkyl group having 3 to 8 carbon atoms, and preferably 3 to 6 carbon atoms. Examples of such cycloalkyl group include a cyclopropyl group, a cyclobutyl group, a cyclopentyl group, a cyclohexyl group, a cycloheptyl group, and a cyclooctyl group.

The “C₁₋₆ alkoxy-C₁₋₆ alkyl group” as used herein means a C₁₋₆ alkyl group substituted with one to three C₁₋₆ alkoxy groups and examples thereof include a methoxymethyl group, a methoxyethyl group, a methoxypropyl group, an ethoxymethyl group, an ethoxyethyl group, a propoxymethyl group, a propoxyethyl group, a butoxymethyl group, a butoxyethyl group, and a 2,2-dimethoxyethyl group.

The “C₆₋₁₀ aryl” as used herein means a monocyclic or a condensed-ring type carbocyclic aryl group having 6 to 10 carbon atoms. Examples of the aryl group include a phenyl group, a naphthyl group, and an azulenyl group.

The “C₆₋₁₀ aryl-C₁₋₆ alkyl group” as used herein means a C₁₋₆ alkyl group substituted with the C₆₋₁₀ aryl group described above. Specific examples thereof include a benzyl group, a phenethyl group, a phenylpropyl group, a phenylbutyl group, a phenylpentyl group, a phenylhexyl group, a naphthylmethyl group, and an azulenylmethyl group.

Examples of the preferred mode of the invention include the followings.

As for R¹ of the formula (1), preferred examples of the C₁₋₆ alkyl group include a C₃₋₅ alkyl group. For example, a n-propyl group, a n-butyl group, and a n-pentyl group are preferable.

As for R¹ in the formula (1), preferred examples of the C₁₋₆ alkoxy group include a C₂₋₅ alkoxy group. For example, a n-propoxy group is preferable.

As for R² in the formula (1), preferred examples of the C₁₋₆ alkoxy group include a C₁₋₄ alkyl group. For example, a methyl group is preferable.

As for R² in the formula (1), preferred examples of the C₃₋₈ cycloalkyl group include a C₃₋₆ cycloalkyl group. For example, a cyclopropyl group, a cyclobutyl group, and a cyclopentyl group are preferable.

As for R³ in the formula (1), preferred examples of the C₁₋₆ alkyl group include a C₁₋₄ alkyl group. For example, a methyl group and an ethyl group are preferable.

As for R³ in the formula (1), preferred examples of the “C₆₋₁₀ aryl-C₁₋₆ alkyl group” include a “C₆₋₁₀ aryl-C₁₋₄ alkyl group”. For example, a benzyl group and a phenethyl group are preferable.

As for R³ in the formula (1), preferred examples of the “C₁₋₆ alkoxy-C₁₋₆ alkyl group” include a “C₁₋₄ alkoxy-C₁₋₄ alkyl group” is preferable. For example, a methoxyethyl group is preferable.

As for R⁵ in the formula (2), preferred examples of the C₁₋₆ alkoxy group include a C₁₋₄ alkoxy group. For example, a methoxy group and an ethoxy group are preferable. The substitution position of R⁵ can be any one of position 4, position 5, and position 6. Position 5 is preferable.

More preferred examples of the compounds that are represented by the formula (1) include a compound that is selected from a group consisting of following compounds:
3-{[2′-(1H-tetrazol-5-yl)biphenyl-4-yl]methyl}-1-benzyl-4-butyl-6-methylpyridin-2(1H)-one,
3-{4′-[(1-benzyl-4-butyl-6-methyl-2-oxo-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-1-benzyl-6-methyl-4-propylpyridin-2(1H)-one,
3-{4′-[(1-benzyl-6-methyl-2-oxo-4-propyl-1,2-dihydropyridin-3-yl)methyl]biphenyl-2-yl}-1,2,4-oxadiazol-5(4H)-one
3-{[2′-(1H-tetrazol-5-yl)biphenyl-4-yl]methyl}-1-benzyl-6-methyl-4-pentylpyridin-2(1H)-one,
3-{4′-[(1-benzyl-6-methyl-2-oxo-4-pentyl-1,2-dihydropyridin-3-yl)methyl]biphenyl-2-yl}-1,2,4-oxadiazol-5(4H)-one
3-{[2′-(1H-tetrazol-5-yl)biphenyl-4-yl]methyl}-4-butyl-6-methyl-1-phenethylpyridin-2(1H)-one,
3-{4′-[(4-butyl-6-methyl-2-oxo-1-phenethyl-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-4-butyl-1-(2-methoxyethyl)-6-methylpyridin-2(1H)-one,
3-{4′-{[4-butyl-1-(2-methoxyethyl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-1-(5-ethoxypyrimidin-2-yl)-6-methylpyridin-2(1H)-one,
3-{4′-{[4-butyl-1-(5-ethoxypyrimidin-2-yl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-1-(5-methoxypyrimidin-2-yl)-6-methylpyridin-2(1H)-one,
3-{4′-{[4-butyl-1-(5-methoxypyrimidin-2-yl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-6-methyl-1-(pyridin-2-yl)pyridin-2(1H)-one,
3-{4′-{[4-butyl-6-methyl-2-oxo-1-(pyridin-2-yl)-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-1-benzyl-6-methyl-4-propoxypyridin-2(1H)-one, and
3-{4′-[(1-benzyl-6-methyl-2-oxo-4-propoxy-1,2-dihydro pyridin-3-yl)methyl]biphenyl-2-yl}-1,2,4-oxadiazol-5(4H)-one.

More preferred examples of the compound represented by the formula (1) include a compound that is selected from a group consisting of following compounds:
3-{[2′-(1H-tetrazol-5-yl)biphenyl-4-yl]methyl}-4-butyl-6-methyl-1-phenethylpyridin-2(1H)-one,
3-{4′-[(4-butyl-6-methyl-2-oxo-1-phenethyl-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-4-butyl-1-(2-methoxyethyl)-6-methylpyridin-2(1H)-one,
3-{4′-{[4-butyl-1-(2-methoxyethyl)-6-methyl-2-oxo-1,2-dihydropyridin-3-yl]methyl}biphenyl-2-yl}-1,2,4-oxadiazol-5(4H)-one,
3-{4′-{[4-butyl-1-(5-ethoxypyrimidin-2-yl)-6-methyl-2-oxo-1,2-dihydropyridin-3-yl]methyl}biphenyl-2-yl}-1,2,4-oxadiazol-5(4H)-one,
3-{4′-{[4-butyl-1-(5-methoxypyrimidin-2-yl)-6-methyl-2-oxo-1,2-dihydropyridin-3-yl]methyl}biphenyl-2-yl}-1,2,4-oxadiazol-5(4H)-one, and
3-{[2′-(1H-tetrazol-5-yl)biphenyl-4-yl]methyl}-4-butyl-6-methyl-1-(pyridin-2-yl)pyridin-2(1H)-one.

If the compound of the invention has geometrical isomers or optical isomers, the invention encompasses all of such isomers. Isolation of these isomers is carried out by an ordinary method.

Salts of the compound represented by the formula (I) are not particularly limited, if they are pharmaceutically acceptable salts. When the compound is processed as an acidic compound, an alkali metal salt or an alkali earth metal salt such as sodium salt, potassium salt, magnesium salt, calcium salt, and the like; and a salt with an organic base such as trimethylamine, triethylamine, pyridine, picoline, N-methylpyrrolidine, N-methylpiperidine, N-methylmorpholine, and the like can be mentioned. When the compound is processed as a basic compound, an acid addition salt and the like including a salt with a mineral acid, for example, hydrochloric acid salt, hydrobromic acid salt, hydroiodic acid salt, sulfuric acid salt, nitric acid salt, phosphoric acid salt and the like; an organic acid addition salt, for example, benzoic acid salt, methanesulfonic acid salt, ethanesulfonic acid salt, benzenesulfonic acid salt, p-toluene sulfonic acid salt, maleic acid salt, fumaric acid salt, tartaric acid salt, citric acid salt, and acetic acid salt; or the like can be mentioned.

Examples of the solvate of the compound represented by the formula (1) or a salt thereof include a hydrate, but not limited thereto.

In addition, compounds which are metabolized in a living body and converted into the compounds represented by the aforementioned formula (I), so called prodrugs, all fall within the scope of the compounds of the invention. Examples of groups which form the prodrugs of the compounds of the invention include the groups described in “Progress in Medicine”, vol. 5, pp. 2157-2161, 1985, Life Science Medica, and the groups described in “Development of Drugs”, vol. 7, Molecular Designs, pp. 163 to 198, 1990, Hirokawa Shoten.

Method for producing compounds represented by the formula (1) or a salt thereof, or a solvate thereof.

The compounds represented by the formula (1), or salts or solvates thereof can be produced according to various known methods, and the production method is not specifically limited. For example, the compounds can be produced according to the following reaction step. Further, when each reaction shown below is performed, functional groups other than the reaction sites may be protected beforehand as required, and deprotected in an appropriate stage. Regarding the conditions for protection and deprotection, it can be carried out with reference to a method that can be generally used (Protective Groups in Organic Synthesis Third Edition, John Wiley & Sons, Inc.). Furthermore, the reaction in each step may be performed by an ordinarily used method, and isolation and purification can be performed by a method suitably selected from conventional methods such as crystallization, recrystallization, chromatography, or the like, or a combination thereof.

[Method for Production of the Compound (1a) and Compound (1b)]

Among the compounds represented by the formula (1) of the invention, the compound (1a) wherein R⁴ is a tetrazolyl group can be produced by reacting the nitrile derivative (5) with an azide compound. Further, the compound (1b) wherein R⁴ is a 5-oxo-1,2,4-oxadiazolyl group can be produced by reacting the nitrile derivative (5) with hydroxylamine to give an amide oxime (6) and subsequently reacting the amide oxime (6) with a carbonyl reagent. The reaction pathway can be expressed with the following chemical reaction scheme.

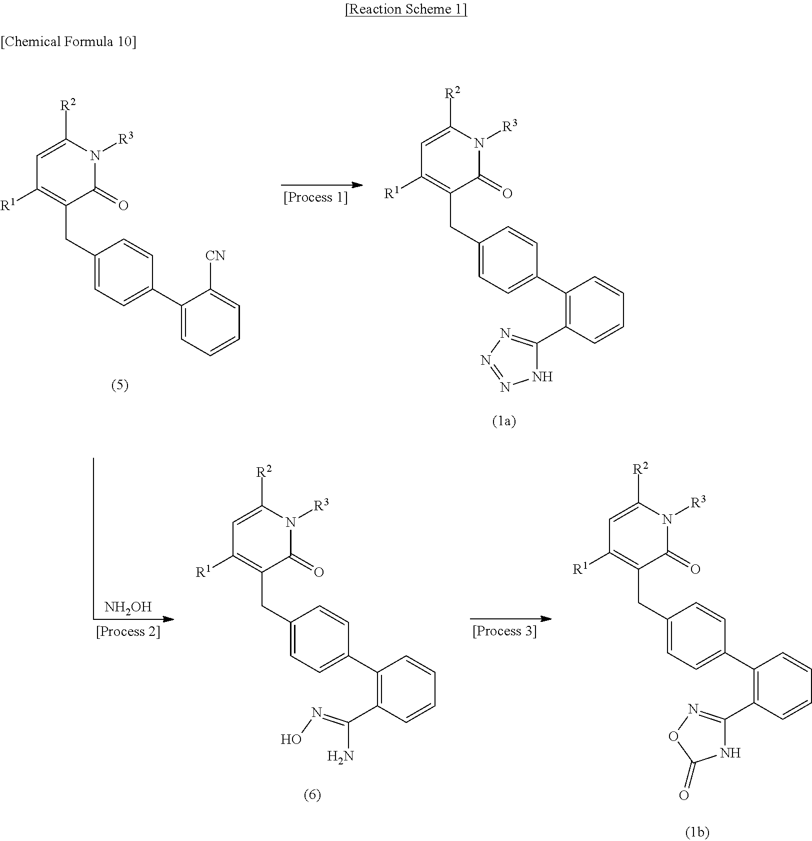

(in the formula, R¹, R², and R³ are as defined above)

[Process 1]

The reaction between the nitrile derivative (5) and an azide compound may be carried out in a solvent. Examples of the azide compound include trimethyltin azide, tributyltin azide, triphenyltin azide, sodium azide, and hydrazoic acid and the like. Further, trimethylsilyl azide may be used in the presence of dibutyltin oxide. The solvent is not specifically limited, and methanol, ethanol, isopropanol, ethyl acetate, isopropyl acetate, toluene, benzene, dioxane, tetrahydrofuran, acetonitrile, propionitrile, N,N-dimethylformamide, N-methylpyrrolidone, and dimethyl sulfoxide and the like may be used either alone or in combination thereof. The reaction condition may vary depending on the reaction materials used. However, the reaction is generally carried out at 0° C. to 180° C., and preferably 50° C. to 120° C. for 1 minute to 2 weeks, and preferably for 1 hour to 3 days to obtain the compound (1a).

[Process 2]

The reaction between the nitrile derivative (5) and hydroxylamine may be carried out in a solvent. The solvent is not specifically limited, and N,N-dimethylformamide, N,N-dimethylacetamide, N-methylpyrrolidone, dimethyl sulfoxide, methanol, ethanol, isopropanol, 1,4-dioxane, and tetrahydrofuran and the like may be used either alone or in combination thereof. When a salt with an acid such as hydroxylamine hydrochloride, hydroxylamine sulfuric acid, hydroxylamine oxalic acid, and the like is used as hydroxylamine, the reaction may be performed in the presence of a suitable base, for example, potassium carbonate, sodium hydrogen carbonate, sodium hydroxide, triethylamine, sodium methoxide, sodium hydride, or the like, in an equivalent amount or a slightly excess amount. The reaction condition may vary depending on the reaction materials used. However, the reaction is generally carried out at 0° C. to 180° C., and preferably 50° C. to 120° C. for 1 minute to 3 days, and preferably for 1 hour to 36 hours to obtain the amide oxime derivative (6).

[Process 3]

Conversion from the amide oxime derivative (6) to the compound (1b) may be carried out in a solvent in the presence of a base using a carbonyl reagent. The solvent is not specifically limited, and 1,2-dichloroethane, chloroform, dichloromethane, ethyl acetate, isopropyl acetate, toluene, benzene, tetrahydrofuran, dioxane, acetonitrile, propionitrile, N,N-dimethylformamide, N,N-dimethylacetamide, N-methylpyrrolidone, diethyl ether and the like may be used either alone or in combination thereof. The base is not specifically limited, and examples thereof include pyridine, N,N-4-dimethylaminopyridine (DMAP), collidine, lutidine, 1,8-diazabicyclo[5.4.0]undec-7-ene (DBU), 1,5-diazabicyclo[3.4.0]non-7-ene (DBN), 1,4-diazabicyclo[2.2.2]octane (DABCO), triethylamine, N,N-diisopropylethylamine, diisopropylpentylamine, trimethylamine, lithium carbonate, sodium carbonate, potassium carbonate, cesium carbonate, sodium hydrogen carbonate, and potassium hydrogen carbonate and the like. The carbonyl reagent is not specifically limited, and 1,1′-carbonyl diimidazole, triphosgene, methyl chlorocarbonate, ethyl chlorocarbonate, and the like may be used. The reaction condition may vary depending on the reaction materials used. However, the reaction is generally carried out at 0° C. to 120° C., and preferably 15° C. to 80° C. for 5 minutes to 3 days, and preferably for 1 hour to 12 hours to obtain the compound (1b).

[Method 1 for Producing Intermediate (5): Compound Wherein R¹ is an Alkyl Group]

The compound represented by the formula (5) in which R¹ is an alkyl group can be produced by reacting a 2-pyrone derivative (7) with an amine derivative (8) to give a 2-pyridone derivative (9), reacting the compound (9) obtained with a compound (10) to give a compound (II), converting the hydroxy group of the compound (II) obtained to a leaving group to yield a compound (12), and reacting the compound (12) with an organo zinc reagent (13) in the presence of a catalyst. The reaction pathway can be expressed with the following chemical reaction scheme.

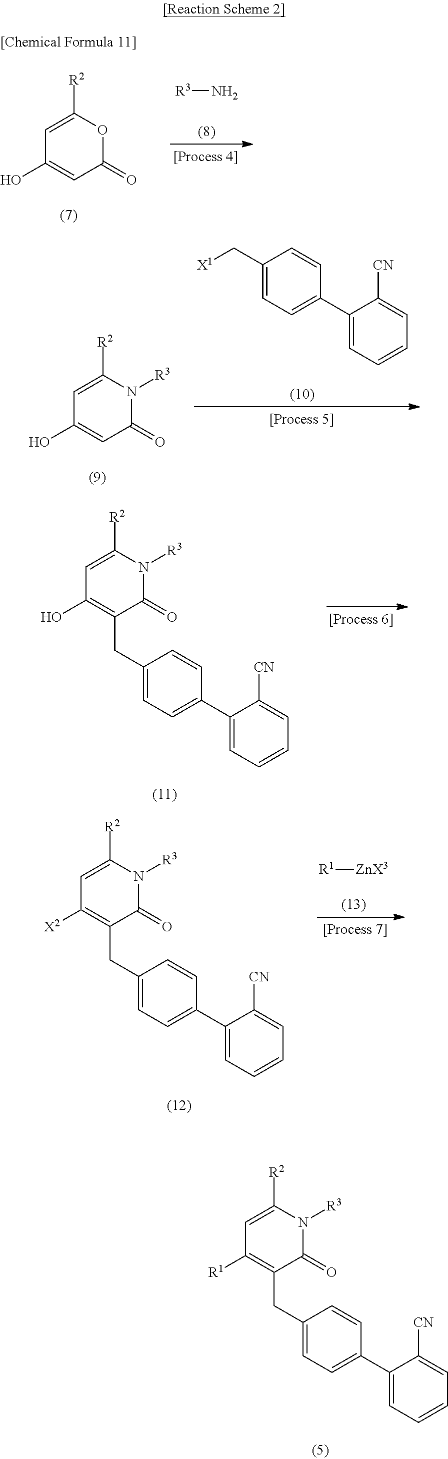

(in the formula, R¹, R², and R³ are as defined above, X¹ and X² represent a leaving group such as a halogen atom, a sulfonyloxy group, or the like, and X³ represents a halogen atom).

### [Process 4]

The process may be performed with reference to a known method like those described by Richard H. et al. [J. Amer. Chem. Soc., 2393-2398 (1956)]. Specifically, the 2-pyrone derivative (7) and the amine derivative (8) may be reacted in a solvent under heating. The solvent is not specifically limited, and dichloromethane, 1,2-dichloroethane, chloroform, N,N-dimethylformamide, N,N-dimethylacetamide, N-methylpyrrolidone, dimethyl sulfoxide, methanol, ethanol, isopropanol, 1,4-dioxane, tetrahydrofuran, water, and the like may be used either alone or in combination thereof. The reaction condition may vary depending on the reaction materials used. However, the reaction is generally carried out at 50° C. to 150° C., and preferably 80° C. to 120° C. for 5 minutes to 3 days, and preferably for 1 hour to 2 days to obtain a 2-pyridone derivative (9).

[Process 5]

The reaction between the 2-pyridone derivative (9) and a compound (10) may be carried out in a solvent in the presence of a base. Further, the compound (10) can be produced with reference to a known method like those described by Devid J. Carini et al. [J. Med. Chem., 34, 2525-2547 (1991)]. The solvent that is used for the process is not specifically limited, and N,N-dimethylformamide, N,N-dimethylacetamide, N-methylpyrrolidone, dimethyl sulfoxide, methanol, ethanol, isopropanol, 1,4-dioxane, tetrahydrofuran, and the like may be used either alone or in combination thereof. The base is not specifically limited, and examples thereof include pyridine, DMAP, collidine, lutidine, DBU, DBN, DABCO, triethylamine, N, N-diisopropylethylamine, diisopropylpentylamine, trimethylamine, lithium carbonate, sodium carbonate, potassium carbonate, cesium carbonate, sodium hydrogen carbonate, potassium hydrogen carbonate, sodium hydride, potassium hydride, and the like. The reaction condition may vary depending on the reaction materials used. However, the reaction is generally carried out at 0° C. to 120° C., and preferably 50° C. to 120° C. for 5 minutes to 3 days, and preferably for 1 hour to 2 days to obtain a compound (II). Further, under the purpose of avoiding a side reaction, lithium halide such as lithium chloride and the like may be added to the present reaction.

[Process 6]

The present process is a process for producing a compound (12) by converting the hydroxy group of the compound (II) to a functional group with leaving property such as halogen, sulfonyloxy, and the like, and it may be carried out in a solvent in the presence of a base. Conversion of a hydroxy group to a halogen atom can be carried out in a solvent using a halogenating agent. Examples of the halogenating agent include phosphorus trichloride, phosphorus pentachloride, phosphorus tribromide, phosphorus oxychloride, phosphorus oxybromide, and the like. As a solvent, dichloromethane, chloroform, diethyl ether, tetrahydrofuran, dioxane, benzene, toluene, xylene, ethyl acetate, acetonitrile, propionitrile, N,N-dimethylformamide, and the like may be used either alone or in combination thereof. Further, under the purpose of promoting the reaction, an organic base such as pyridine, triethylamine, and N,N-diisopropylethylamine, and the like can be also added as a catalyst. The reaction condition includes −80° C. to 180° C., and preferably 0° C. to 100° C. for 1 minute to 3 days, and preferably for 30 minutes to 24 hours to obtain a compound (12) wherein X² is a halogen atom.

Conversion of a hydroxy group to a sulfonyloxy group can be carried out in a solvent in the presence of a base by reacting the compound (II) with sulfonic anhydride or sulfonic halide, for example. As a solvent, dichloromethane, chloroform, diethyl ether, tetrahydrofuran, dioxane, benzene, toluene, xylene, ethyl acetate, acetonitrile, propionitrile, and N, N-dimethylformamide, and the like may be used either alone or in combination thereof, for example. Examples of sulfonic anhydride include methane sulfonic anhydride, benzene sulfonic anhydride, trifluoromethane sulfonic anhydride, and the like. Examples of the sulfonic halide include methanesulfonyl chloride, p-toluenesulfonyl chloride, and the like. Examples of the base include an organic base such as pyridine, triethylamine, N,N-diisopropylethylamine, and the like and an inorganic base such as potassium hydrogen carbonate, sodium hydrogen carbonate, potassium carbonate, sodium carbonate, calcium carbonate, and the like. The reaction condition may vary depending on the reaction materials used. However, the reaction is generally carried out at −80° C. to 180° C., and preferably 0° C. to 100° C. for 1 minute to 3 days, and preferably for 30 minutes to 24 hours to obtain a compound (12) wherein X² is a sulfonyloxy group.

### [Process 7]

The reaction between a compound (12) and an organo zinc compound (13) may be carried out in a solvent in the presence of a catalyst. As a solvent, benzene, toluene, xylene, diethyl ether, tetrahydrofuran, dimethoxyethane, dioxane, acetonitrile, N,N-dimethylformamide, N-methylpiperidone, methanol, ethanol, water, and the like may be used either alone or in combination thereof, for example. Examples of the catalyst include palladium (0) tetrakis(triphenylphosphine), dipalladium (0) tris(bibenzylidene acetone), palladium (II) acetate, palladium (II) chloride, palladium (II) dichloro[1,2-bis(diphenylphosphino)ethane], palladium (II) dichloro[1,2-bis(diphenylphosphino)butane], palladium (II) dichloro[1,1′-bis(diphenylphosphino)ferrocene], and the like. Further, if necessary, a ligand such as tri(tert-butyl)phosphine, triphenylphosphine, tri(o-tolyl)phosphine, tri(2-furyl)phosphine, 2,2′-bis(diphenylphosphino)-1,1′-binaphthyl, 1,2-bis(diphenylphosphino)ethane, and the like may be combined with the catalyst.

The reaction condition may vary depending on the reaction materials used. However, the reaction is generally carried out at 20° C. to 180° C., and preferably 20° C. to 100° C. for 1 minute to 3 days, and preferably for 1 hour to 24 hours to obtain a compound (5).

A compound (9) wherein R² is a methyl group may be also produced by using a diketene (14) as a starting material. The reaction pathway can be expressed with the following chemical reaction scheme.

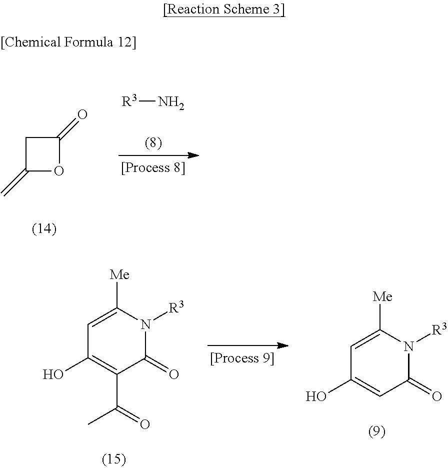

### [Process 8]

The process may be performed with reference to a method described by Tetsuzo Kato et al. [Chem. Pharm. Bull., 20(1), 133-141 (1972)] or a similar method. Specifically, the reaction between a diketene (14) and an amine derivative (8) may be performed in a solvent under heating. As a solvent, dichloromethane, chloroform, diethyl ether, tetrahydrofuran, dioxane, benzene, toluene, xylene, ethyl acetate, acetonitrile, propionitrile, N,N-dimethylformamide, acetic acid, and the like may be used either alone or in combination thereof. The reaction condition may vary depending on the reaction materials used. However, the reaction is generally carried out at 50° C. to 180° C., and preferably 60° C. to 120° C. for 1 minute to 48 hours, and preferably for 10 minutes to 24 hours to obtain a compound (15).

### [Process 9]

By reacting the compound (15) under heating in the presence of an acid, the 2-pyridone derivative (9) can be produced. As an acid, hydrochloric acid, sulfuric acid, nitric acid, or the like is preferably used not only as a solvent but also as a reagent. The reaction condition may vary depending on the reaction materials used. However, the reaction is generally carried out at 50° C. to 180° C., and preferably 60° C. to 150° C. for 1 minute to 12 hours, and preferably for 1 minute to 6 hours to obtain the 2-pyridone derivative (9).

[Method 2 for producing intermediate (5): compound wherein R¹ is an alkyl group]

The compound represented by the formula (5) wherein R¹ is an alkyl group can also be produced by reacting a 2-pyrone derivative (7) with the compound (10) to give a compound (16), converting the hydroxy group of the compound (16) obtained to a leaving group, thus yielding a compound (17), reacting the compound (17) obtained with an organo zinc reagent (13) in the presence of a catalyst to give a compound (18), and reacting the compound (18) with an amine derivative (8). The reaction pathway can be expressed with the following chemical reaction scheme.

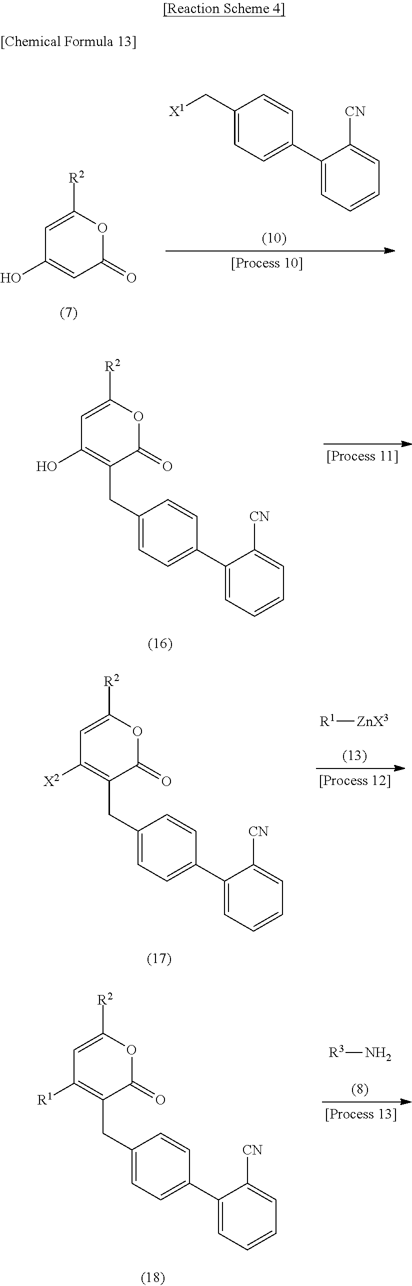

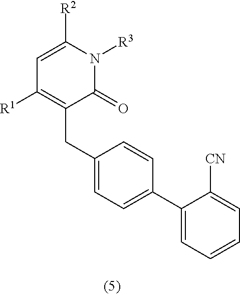

(in the formula, R¹, R², R³, X¹, X², and X³ are as defined above).

### [Process 10]

The present process is a process for producing a compound (16) by reacting the 2-pyrone derivative (7) and the compound (10) in a solvent in the presence of a base, and the reaction can be carried out by using the method described in the Process 5 above.

### [Process 11]

The present process is a process for producing a compound (17) by converting the hydroxy group of the compound (16) to a leaving group such as halogen, sulfonyloxy, and the like, and the reaction can be carried out by using the method described in the Process 6 above.

### [Process 12]

The present process is a process for producing a compound (18) by reacting a compound (17) and an organo zinc compound (13) in a solvent in the presence of a catalyst, and the reaction can be carried out by using the method described in the Process 7 above.

### [Process 13]

The present process is a process for producing an intermediate (5) by reacting a compound (18) and the amine derivative (8) in a solvent under heating, and the reaction can be carried out by using the method described in the Process 4 above.

### [Method 3 for Producing Intermediate (5): Compound Wherein R¹ is an Alkoxy Group]

The compound represented by the formula (5′) wherein R¹ is an alkoxy group can be produced by reacting a compound (II), which can be produced according to the Process 5 described above, with a compound (19) in a solvent in the presence of a base. The reaction pathway can be expressed with the following chemical reaction scheme.

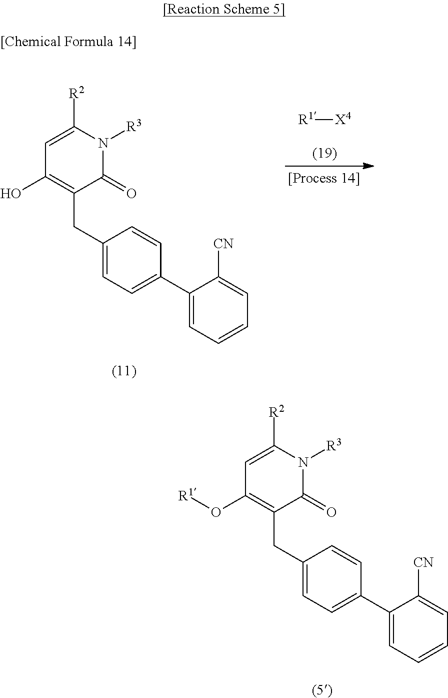

(in the formula, R¹, R², and R³ are as defined above, R^{1,} represents a C₁₋₆ alkyl group, and X⁴ represents a leaving group such as a halogen atom, a sulfonyloxy group, a hydroxy group, or the like).

### [Process 14]

When a compound (19) wherein X⁴ is a halogen atom or a sulfonyloxy group is used, the process can be carried out in a solvent in the presence of a base according to a commonly used alkylation method for a hydroxy group. As a solvent, chloroform, dichloromethane, tetrahydrofuran, toluene, dioxane, methanol, ethanol, ethyl acetate, acetonitrile, propionitrile, water, and the like may be used either alone or in combination thereof. Examples of the base include pyridine, triethylamine, N, N-diisopropylethylamine, potassium carbonate, sodium carbonate, potassium hydroxide, sodium hydroxide, lithium hydroxide, lithium hydride, sodium hydride, potassium hydride, and the like. The reaction condition includes −80° C. to 180° C., and preferably −30° C. to 130° C. for 1 minute to 5 days, and preferably for 15 minutes to 3 days. Further, when a compound (19) wherein X⁴ is a hydroxyl group is used, the compound (5′) can also be produced by reacting the compound (II) with the compound (19) in a solvent in the presence of a phosphine reagent and an azo reagent according to a method that is known as Mitsunobu reaction (Synthesis, 1-28 (1981)). Examples of the phosphine reagent include trialkylphosphines such as trimethylphosphine, triethylphosphine, tripropylphosphine, triisopropylphosphine, tributylphosphine, triisobutylphosphine, tricyclohexylphosphine, and the like and triarylphosphines such as triphenylphosphine. The azo reagent is not specifically limited, and examples thereof include diethyl azodicarboxylate (DEAD), diisopropyl azodicarboxylate (DIAD), di-tert-butyl azodicarboxylate (DBAD), 1,1-(azodicarbonyl)piperidine (ADDP), 1,1′-azobis(N, N′-diisopropyl formamide) (TIPA), 1,6-dimethyl-1,5,7-hexahydro-1,4,6-tetrazocine-2,5-dione (DHAD), and the like. Further, ethylene dicarboxylic acid reagent may be used instead of an azo reagent. Examples of the ethylene dicarboxylic acid include dimethyl maleic acid, diethyl maleic acid, dimethyl fumaric acid, and diethyl fumaric acid, and the like. As a solvent, dichloromethane, chloroform, tetrahydrofuran, diethyl ether, dioxane, benzene, toluene, xylene, methanol, ethanol, N,N-dimethylformamide, and the like may be used either alone or in combination thereof. The reaction condition includes −80° C. to 100° C., and preferably −30° C. to 60° C. for 1 minute to 5 days, and preferably for 15 minutes to 1 day.

If necessary, the intermediates and target compounds that are obtained from each of the reaction above can be isolated and purified by a purification method that is generally used in a field of organic synthesis chemistry, e.g., filtration, extraction, washing, drying, concentration, recrystallization, various chromatographic methods, and the like. Furthermore, the intermediates may be used for the next reaction without any specific purification.

Various isomers may be isolated by applying a general method based on a difference in physicochemical properties among the isomers. For example, a racemic mixture may be resolved into an optically pure isomer by common racemic resolution like optical resolution by which a diastereomer salt is formed with a common optically active acid like tartaric acid or a method of using optically active chromatography. Further, a mixture of diastereomers can be resolved by fractional crystallization or various chromatographic methods, for example. Furthermore, an optically active compound can be also produced by using an appropriate starting compound that is optically active.

The compound (1) obtained may be converted into a salt according to a common method. Furthermore, it may be converted into a solvate with a solvent like a solvent for reaction or a solvent for recrystallization, or into a hydrate.

Examples of a dosage form of the pharmaceutical agent containing the compounds of the invention, salts or solvates thereof as an effective component include, for example, those for oral administration such as tablet, capsule, granule, powder, syrup, or the like and those for parenteral administration such as intravenous injection, intramuscular injection, suppository, inhalant, transdermal preparation, eye drop, nasal drop, or the like. In order to prepare a pharmaceutical preparation in the various dosage forms, the effective component may be used alone, or may be used in appropriate combination with other pharmaceutically acceptable carriers such as excipients, binders, extending agents, disintegrating agents, surfactants, lubricants, dispersing agents, buffering agents, preservatives, corrigents, perfumes, coating agents, diluents, and the like to give a pharmaceutical composition.

Although the administration amount of the pharmaceutical agent of the invention may vary depending on the weight, age, sex, symptoms, and the like of a patient, in terms of the compound represented by the general formula (1), generally 0.1 to 1000 mg, especially 1 to 300 mg, may be administered orally or parenterally at one time or several times as divided portions per day for an adult.

### EXAMPLES

Hereinbelow, the invention will be explained in greater detail with reference to examples. However, the invention is not limited to these examples. The abbreviations used in the examples have the following meanings.

s: singlet

d: doublet

t: triplet

q: quartet

m: multiplet

br: broad

brs: broad singlet

J: coupling constant

Hz: hertz

CDCl₃: deuterated chloroform

DMSO-d₆: deuterated dimethyl sulfoxide

¹H-NMR: proton nuclear magnetic resonance

### Example 1

Production of 3-{[2′-(1H-tetrazol-5-yl)biphenyl-4-yl]methyl}-1-benzyl-4-butyl-6-methylpyridin-2(1H)-one

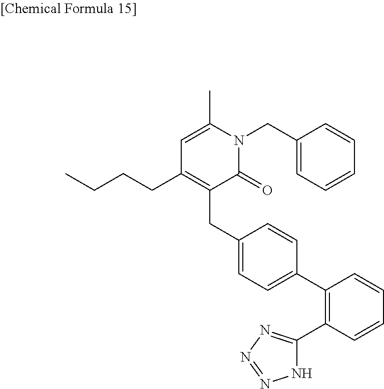

Process 1:

Benzylamine (3.3 mL, 30 mmol) was added to a water (20 mL) solution of 4-hydroxy-6-methyl-2-pyrone (3.8 g, 30 mmol) at room temperature and stirred overnight while being heated under reflux. After cooling to room temperature, the precipitated solids were collected by filtration to obtain 1-benzyl-4-hydroxy-6-methylpyridin-2(1H)-one (3.7 g, 57%) as a pale yellow solid.

¹H-NMR (CD₃OD) δ:

2.25 (3H, s), 4.86 (2H, s), 5.82 (1H, d, J=3 Hz), 5.97 (1H, d, J=3 Hz), 7.08-7.14 (2H, m), 7.21-7.35 (3H, m).

Process 2:

Lithium chloride (212 mg, 5 mmol), N,N-diisopropylethylamine (1.7 mL, 10 mmol), and 4′-bromo methyl-2-cyano biphenyl (1.6 g, 6 mmol) were added to a N,N-dimethylformamide (30 mL) solution of 1-benzyl-4-hydroxy-6-methylpyridin-2(1H)-one (1.1 g, 5 mmol) at room temperature and stirred overnight at 70° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (chloroform/methanol=10:1) to obtain 4′-[(1-benzyl-4-hydroxy-6-methyl-2-oxo-1,2-dihydropyridin-3-yl)methyl]biphenyl-2-carbonitrile (540 mg, 27%) as a pale yellow solid.

¹H-NMR (CD₃OD) δ:

2.25 (3H, s), 3.94 (2H, s), 5.36 (2H, s), 6.04 (1H, s), 7.05-7.34 (5H, m), 7.41-7.58 (6H, m), 7.70 (1H, dt, J=1, 8 Hz), 7.79 (1H, dt, J=1, 8 Hz).

Process 3:

Pyridine (377 μL, 4.8 mmol) and trifluoromethane sulfonic anhydride (669 μL, 4.0 mmol) were added to a dichloromethane (10 mL) solution of 4′-[(1-benzyl-4-hydroxy-6-methyl-2-oxo-1,2-dihydropyridin-3-yl)methyl]biphenyl-2-carbonitrile (540 mg, 1.3 mmol) at room temperature and stirred for 2 hours at room temperature. The reaction solution was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain pale yellow oil. Under argon atmosphere, a n-butyl zinc bromide (0.5 mol/L tetrahydrofuran solution, 9 mL, 4.5 mmol) was added to a toluene (25 mL) solution of the pale yellow oil obtained from the above and dichloro[1,1′-bis(diphenylphosphino)ferrocene] palladium (II) dichloromethane complex (181 mg, 0.23 mmol) at 0° C. and stirred for 1.5 hours at 90° C. The reaction mixture was added ethyl acetate and filtered through a pad of celite. The filtrate was added water to separate the organic layer, and the aqueous layer was extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain 4′-[(1-benzyl-4-butyl-6-methyl-2-oxo-1,2-dihydropyridin-3-yl)methyl]biphenyl-2-carbonitrile (413 mg, 70%) as pale yellow oil.

¹H-NMR (CDCl₃) δ:

0.91 (3H, t, J=7 Hz), 1.32-1.42 (2H, m), 1.44-1.54 (2H, m), 2.26 (3H, s), 2.51 (2H, t, J=8 Hz), 4.07 (2H, s), 5.35 (2H, s), 5.95 (1H, s), 7.14 (2H, d, J=7 Hz), 7.21-7.34 (3H, m), 7.36-7.51 (6H, m), 7.60 (1H, dt, J=1, 8 Hz), 7.73 (1H, dt, J=1, 8 Hz).

Process 4:

Trimethylsilyl azide (264 μL, 2.0 mmol) and dibutyltin oxide (5 mg, 0.020 mmol) were added to a toluene (1.5 mL) solution of 4′-[(1-benzyl-4-butyl-6-methyl-2-oxo-1,2-dihydropyridin-3-yl)methyl]biphenyl-2-carbonitrile (18 mg, 0.040 mmol) and heated under reflux for 16 hours under argon atmosphere. The reaction solvent was distilled off, and the residues obtained were separated and purified by silica gel column chromatography (chloroform:methanol=20:1) to obtain 3-{[2′-(1H-tetrazol-5-yl)biphenyl-4-yl]methyl}-1-benzyl-4-butyl-6-methylpyridin-2(1H)-one (8 mg, 40%) as pale yellow oil.

¹H-NMR (CDCl₃) δ:

0.90 (3H, t, J=7 Hz), 1.30-1.40 (2H, m), 1.45-1.55 (2H, m), 2.23 (3H, s), 2.48 (2H, t, J=8 Hz), 3.85 (2H, s), 5.18 (2H, s), 6.01 (1H, s), 6.90 (2H, d, J=8 Hz), 6.95 (2H, d, J=8 Hz), 7.00 (2H, d, J=7 Hz), 7.17-7.52 (6H, m), 7.88 (1H, d, J=8 Hz).

### Example 2

Production of 3-{4′-[(1-benzyl-4-butyl-6-methyl-2-oxo-1,2-dihydropyridin-3-yl)methyl]biphenyl-2-yl}-1,2,4-oxadiazol-5(4H)-one

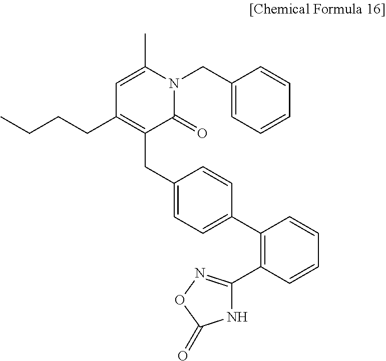

Process 1:

Sodium hydrogen carbonate (948 mg, 11.29 mmol) was added to a dimethyl sulfoxide (5 mL) solution of hydroxylamine hydrochloride (654 mg, 9.40 mmol) and stirred for 1 hour at 40° C. The reaction mixture was added dimethyl sulfoxide solution (3 mL) of 4′-[(1-benzyl-4-butyl-6-methyl-2-oxo-1,2-dihydropyridin-3-yl)methyl]biphenyl-2-carbonitrile (168 mg, 0.38 mmol) obtained in the Process 3 of the Example 1 and stirred for 16 hours at 90° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain pale yellow oil.

Process 2:

1,1-carbonyl diimidazole (114 mg, 0.70 mmol) and 1,8-diazabicyclo[5.4.0]undec-7-ene (105 μL, 0.70 mmol) were added to an N,N-dimethylformamide solution (1.5 mL) of the pale yellow oil obtained and stirred for 2 hours at room temperature. After completion of the reaction, the reaction solution was added water and extracted with ethyl acetate. The organic layer was combined, washed with water and brine, dried over anhydrous sodium sulfate, and concentrated in vacuo. The residues obtained were purified by silica gel column chromatography (ethyl acetate) to obtain 3-{4′-[(1-benzyl-4-butyl-6-methyl-2-oxo-1,2-dihydropyridin-3-yl)methyl]biphenyl-2-yl}-1,2,4-oxadiazol-5(4H)-one (79 mg, 51%) as pale yellow oil.

¹H-NMR (CDCl₃) δ:

0.91 (3H, t, J=7 Hz), 1.31-1.41 (2H, m), 1.46-1.56 (2H, m), 2.22 (3H, s), 2.51 (2H, t, J=8 Hz), 3.89 (2H, s), 5.16 (2H, s), 6.00 (1H, s), 6.98 (2H, d, J=7 Hz), 7.08-7.18 (4H, m), 7.19-7.41 (5H, m), 7.51 (1H, dt, J=1, 8 Hz), 7.88 (1H, d, J=8 Hz).

### Example 3

Production of 3-{[2′-(1H-tetrazol-5-yl)biphenyl-4-yl]methyl}-1-benzyl-6-m ethyl-4-propylpyridin-2(1H)-one

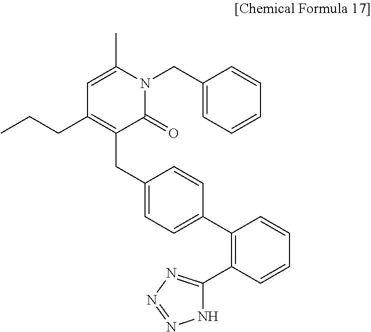

Process 1:

4′-[(1-benzyl-6-methyl-2-oxo-4-propyl-1,2-dihydropyridin-3-yl)methyl]biphenyl-2-carbonitrile was obtained according to the same reaction and treatment as the Process 3 of the Example 1 by using n-propyl zinc bromide instead of the n-butyl zinc bromide.

¹H-NMR (CDCl₃) δ:

0.96 (3H, t, J=7 Hz), 1.48-1.61 (2H, m), 2.25 (3H, s), 2.50 (2H, t, J=8 Hz), 4.08 (2H, s), 5.35 (2H, s), 5.95 (1H, s), 7.05 (2H, d, J=7 Hz), 7.20-7.34 (3H, m), 7.36-7.50 (6H, m), 7.60 (1H, dt, J=1, 8 Hz), 7.72 (1H, dt, J=1, 8 Hz).

Process 2:

3-{[2′-(1H-tetrazol-5-yl)biphenyl-4-yl]methyl}-1-benzyl-6-m ethyl-4-propylpyridin-2(1H)-one was obtained according to the same reaction and treatment in the Process 4 of the Example 1 by using 4′-[(1-benzyl-6-methyl-2-oxo-4-propyl-1,2-dihydropyridin-3-yl)methyl]biphenyl-2-carbonitrile instead of the 4′-[(1-benzyl-4-butyl-6-methyl-2-oxo-1,2-dihydropyridin-3-yl)methyl]biphenyl-2-carbonitrile.

¹H-NMR (CDCl₃) δ:

0.95 (3H, t, J=7 Hz), 1.49-1.61 (2H, m), 2.23 (3H, s), 2.47 (2H, t, J=8 Hz), 3.85 (2H, s), 5.18 (2H, s), 6.02 (1H, s), 6.88-6.96 (3H, m), 7.00 (2H, d, J=7 Hz), 7.19-7.51 (7H, m), 7.93 (1H, d, J=7 Hz).

### Example 4

Production of 3-{4′-[(1-benzyl-6-methyl-2-oxo-4-propyl-1,2-dihydropyridin-3-yl)methyl]biphenyl-2-yl}-1,2,4-oxadiazol-5(4H)-one

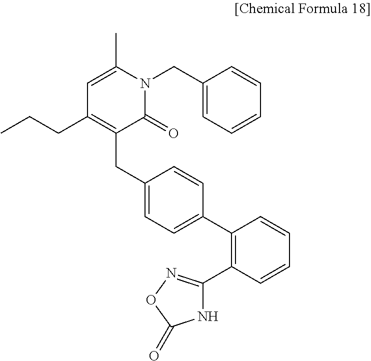

3-{4′-[(1-benzyl-6-methyl-2-oxo-4-propyl-1,2-dihydropyridin-3-yl)methyl]biphenyl-2-yl}-1,2,4-oxadiazol-5(4H)-one was obtained according to the same reaction and treatment in the Processes 1 and 2 of the Example 2 by using the 4′-[(1-benzyl-6-methyl-2-oxo-4-propyl-1,2-dihydropyridin-3-yl)methyl] biphenyl-2-carbonitrile obtained in the Process 1 of the Example 3 instead of the 4′-[(1-benzyl-4-butyl-6-methyl-2-oxo-1,2-dihydropyridin-3-yl)methyl]biphenyl-2-carbonitrile.

### Example 5

Production of 3-{[2′-(1H-tetrazol-5-yl)biphenyl-4-yl]methyl}-1-benzyl-6-m ethyl-4-pentylpyridin-2(1H)-one

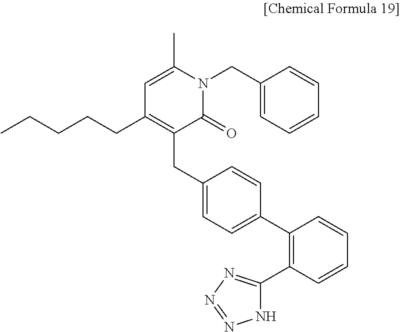

Process 1:

4′-[(1-benzyl-6-methyl-2-oxo-4-pentyl-1,2-dihydropyridin-3-yl)methyl]biphenyl-2-carbonitrile was obtained according to the same reaction and treatment as the Process 3 of the Example 1 by using n-pentyl zinc bromide instead of the n-butyl zinc bromide.

¹H-NMR (CDCl₃) δ:

0.88 (3H, t, J=7 Hz), 1.28-1.37 (4H, m), 1.46-1.56 (2H, m), 2.25 (3H, s), 2.51 (2H, t, J=8 Hz), 4.07 (2H, s), 5.35 (2H, s), 5.95 (1H, s), 7.14 (2H, d, J=8 Hz), 7.20-7.33 (3H, m), 7.35-7.50 (6H, m), 7.60 (1H, dt, J=1, 8 Hz), 7.73 (1H, dt, J=1, 8 Hz).

Process 2:

3-{[2′-(1H-tetrazol-5-yl)biphenyl-4-yl]methyl}-1-benzyl-6-methyl-4-pentylpyridin-2(1H)-one was obtained according to the same reaction and treatment as the Process 4 of the Example 1 by using
4′-[(1-benzyl-6-methyl-2-oxo-4-pentyl-1,2-dihydropyridin-3-yl)methyl]biphenyl-2-carbonitrile instead of the
4′-[(1-benzyl-4-butyl-6-methyl-2-oxo-1,2-dihydropyridin-3-yl)methyl]biphenyl-2-carbonitrile.

¹H-NMR (CDCl₃) δ:

0.88 (3H, t, J=7 Hz), 1.22-1.39 (4H, m), 1.50-1.64 (2H, m), 2.30 (3H, s), 2.56 (2H, t, J=8 Hz), 3.98 (2H, s), 5.32 (2H, s), 6.11 (1H, s), 6.97-7.11 (3H, m), 7.16-7.56 (7H, m), 7.76-7.86 (1H, m), 7.66-8.11 (2H, m).

### Example 6

Production of 3-{4′-[(1-benzyl-6-methyl-2-oxo-4-pentyl-1,2-dihydropyridin-3-yl)methyl]biphenyl-2-yl}-1,2,4-oxadiazol-5(4H)-one

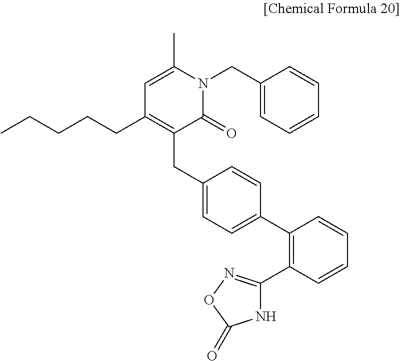

3-{4′-[(1-benzyl-6-methyl-2-oxo-4-pentyl-1,2-dihydropyridin-3-yl)methyl]biphenyl-2-yl}-1,2,4-oxadiazol-5(4H)-one was obtained according to the same reaction and treatment as the Processes 1 and 2 of the Example 2 by using the 4′-[(1-benzyl-6-methyl-2-oxo-4-pentyl-1,2-dihydropyridin-3-yl)methyl] biphenyl-2-carbonitrile obtained in the Process 1 of the Example 5 instead of the 4′-[(1-benzyl-4-butyl-6-methyl-2-oxo-1,2-dihydropyridin-3-yl)methyl]biphenyl-2-carbonitrile.

### Example 7

Production of 3-{[2′-(1H-tetrazol-5-yl)biphenyl-4-yl]methyl}-4-butyl-6-methyl-1-phenethylpyridin-2(1H)-one

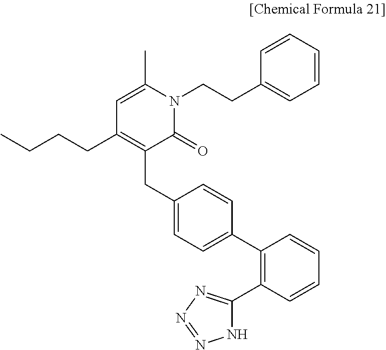

Process 1:

4-hydroxy-6-methyl-1-phenethylpyridin-2(1H)-one was obtained according to the same reaction and treatment as the Process 1 of the Example 1 by using 2-phenylethylamine instead of the benzylamine.

¹H-NMR (CD₃OD) δ:

2.11 (3H, s), 2.96 (2H, t, J=7 Hz), 4.16 (2H, t, J=7 Hz), 5.77 (1H, d, J=3 Hz), 5.84 (1H, d, J=3 Hz), 7.16-7.30 (5H, m).

Process 2:

4′-[(4-hydroxy-6-methyl-2-oxo-1-phenethyl-1,2-dihydro pyridin-3-yl)methyl]biphenyl-2-carbonitrile was obtained according to the same reaction and treatment as the Process 2 of the Example 1 by using 4-hydroxy-6-methyl-1-phenethylpyridin-2(1H)-one instead of the 1-benzyl-4-hydroxy-6-methylpyridin-2(1H)-one.

¹H-NMR (CD₃OD) δ:

2.15 (3H, s), 2.98 (2H, t, J=8 Hz), 3.93 (2H, s), 4.19 (2H, t, J=8 Hz), 5.91 (1H, s), 7.14-7.31 (5H, m), 7.39-7.88 (8H, m).

Process 3:

4′-[(4-butyl-6-methyl-2-oxo-1-phenethyl-1,2-dihydropyridin-3-yl)methyl] biphenyl-2-carbonitrile was obtained according to the same reaction and treatment as the Process 3 of the Example 1 by using
4′-[(4-hydroxy-6-methyl-2-oxo-1-phenethyl-1,2-dihydropyridin-3-yl)methyl]biphenyl-2-carbonitrile instead of the 4′-[(1-benzyl-4-hydroxy-6-methyl-2-oxo-1,2-dihydropyridin-3-yl)methyl]biphenyl-2-carbonitrile.

¹H-NMR (CDCl₃) δ:

0.90 (3H, t, J=7 Hz), 1.30-1.40 (2H, m), 1.42-1.51 (2H, m), 2.19 (3H, s), 2.48 (2H, t, J=8 Hz), 3.02 (2H, t, J=8 Hz), 4.07 (2H, s), 4.21 (2H, t, J=8 Hz), 5.88 (1H, s), 7.19-7.32 (5H, m), 7.34-7.41 (3H, m), 7.43-7.49 (3H, m), 7.59 (1H, dt, J=1, 8 Hz), 7.71 (1H, d, J=8 Hz).

Process 4:

3-{[2′-(1H-tetrazol-5-yl)biphenyl-4-yl]methyl}-4-butyl-6-methyl-1-phenethylpyridin-2(1H)-one was obtained according to the same reaction and treatment as the Process 4 of the Example 1 by using 4′-[(4-butyl-6-methyl-2-oxo-1-phenethyl-1,2-dihydropyridin-3-yl)methyl]biphenyl-2-carbonitrile instead of the 4′-[(1-benzyl-4-butyl-6-methyl-2-oxo-1,2-dihydropyridin-3-yl)methyl]biphenyl-2-carbonitrile.

¹H-NMR (CDCl₃) δ:

0.88 (3H, t, J=7 Hz), 1.32-1.39 (2H, m), 1.40-1.46 (2H, m), 2.08 (3H, s), 2.43 (2H, t, J=8 Hz), 2.87 (2H, t, J=8 Hz), 3.84 (2H, s), 4.08 (2H, t, J=8 Hz), 5.93 (1H, s), 6.92 (4H, brs), 7.03-7.09 (2H, m), 7.15-7.23 (2H, m), 7.27-7.49 (4H, m), 7.86 (1H, d, J=7 Hz).

### Example 8

Production of 3-{4′-[(4-butyl-6-methyl-2-oxo-1-phenethyl-1,2-dihydropyridin-3-yl)methyl]biphenyl-2-yl}-1,2,4-oxadiazol-5(4H)-one

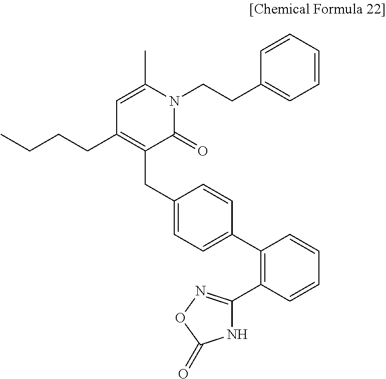

3-{4′-[(4-butyl-6-methyl-2-oxo-1-phenethyl-1,2-dihydropyridin-3-yl)methyl]biphenyl-2-yl}-1,2,4-oxadiazol-5(4H)-one was obtained according to the same reaction and treatment as the Processes 1 and 2 of the Example 2 by using the 4′-[(4-butyl-6-methyl-2-oxo-1-phenethyl-1,2-dihydropyridin-3-yl)methyl]biphenyl-2-carbonitrile obtained in the Process 3 of the Example 7 instead of the 4′-[(1-benzyl-4-butyl-6-methyl-2-oxo-1,2-dihydropyridin-3-yl)methyl]biphenyl-2-carbonitrile.

¹H-NMR (CDCl₃) δ:

0.93 (3H, t, J=7 Hz), 1.33-1.47 (4H, m), 2.19 (3H, s), 2.52 (2H, t, J=8 Hz), 2.96 (2H, t, J=8 Hz), 3.96 (2H, s), 4.14 (2H, t, J=8 Hz), 5.95 (1H, s), 7.14-7.31 (9H, m), 7.38-7.49 (2H, m), 7.54-7.62 (1H, m), 7.79 (1H, d, J=8 Hz).

### Example 9

Production of 3-{[2′-(1H-tetrazol-5-yl)biphenyl-4-yl]methyl}-4-butyl-1-(2-methoxyethyl)-6-methylpyridin-2(1H)-one

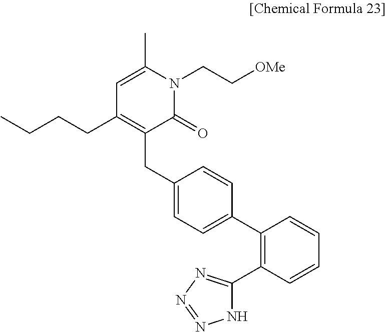

Process 1:

4-hydroxy-1-(2-methoxyethyl)-6-methylpyridin-2(1H)-one was obtained according to the same reaction and treatment as the Process 1 of the Example 1 by using 2-methoxyethylamine instead of the benzylamine.

¹H-NMR (CD₃OD) δ:

0.88 (3H, t, J=7 Hz), 1.28-1.39 (2H, m), 1.42-1.52 (2H, m), 2.35 (3H, s), 2.43 (2H, t, J=8 Hz), 3.16 (3H, s), 3.47 (2H, brs), 3.80 (2H, s), 4.04 (2H, brs), 5.99 (1H, s), 6.89 (4H, brs), 7.29-7.46 (3H, m), 7.87 (1H, d, J=7 Hz).

Process 2:

4′-{[4-hydroxy-1-(2-methoxyethyl)-6-methyl-2-oxo-1,2-dihydropyridin-3-yl]methyl}biphenyl-2-carbonitrile was obtained according to the same reaction and treatment as the Process 2 of the Example 1 by using 4-hydroxy-1-(2-methoxyethyl)-6-methylpyridin-2(1H)-one instead of the 1-benzyl-4-hydroxy-6-methylpyridin-2(1H)-one.

¹H-NMR (CD₃OD) δ:

0.90 (3H, t, J=7 Hz), 1.31-1.40 (2H, m), 1.42-1.53 (2H, m), 2.40 (3H, s), 2.48 (2H, t, J=8 Hz), 3.31 (3H, s), 3.69 (2H, t, J=5 Hz), 4.02 (2H, s), 4.20 (2H, t, J=5 Hz), 5.92 (1H, s), 7.32-7.49 (6H, m), 7.60 (1H, dt, J=1, 8 Hz), 7.72 (1H, dd, J=1, 8 Hz).

Process 3:

4′-{[4-butyl-1-(2-methoxyethyl)-6-methyl-2-oxo-1,2-dihydropyridin-3-yl)methyl]biphenyl-2-carbonitrile was obtained according to the same reaction and treatment as the Process 3 of the Example 1 by using 4′-{[4-hydroxy-1-(2-methoxyethyl)-6-methyl-2-oxo-1,2-dihydropyridin-3-yl]methyl}biphenyl-2-carbonitrile instead of the 4′-[(1-benzyl-4-hydroxy-6-methyl-2-oxo-1,2-dihydropyridin-3-yl)methyl]biphenyl-2-carbonitrile.

¹H-NMR (CDCl₃) δ:

0.90 (3H, t, J=7 Hz), 1.31-1.40 (2H, m), 1.42-1.53 (2H, m), 2.40 (3H, s), 2.48 (2H, t, J=8 Hz), 3.31 (3H, s), 3.69 (2H, t, J=5 Hz), 4.02 (2H, s), 4.20 (2H, t, J=5 Hz), 5.92 (1H, s), 7.32-7.49 (6H, m), 7.60 (1H, dt, J=1, 8 Hz), 7.72 (1H, dd, J=1, 8 Hz).

Process 4:

3-{[2′-(1H-tetrazol-5-yl)biphenyl-4-yl]methyl}-4-butyl-1-(2-methoxyethyl)-6-methylpyridin-2(1H)-one was obtained according to the same reaction and treatment as the Process 4 of the Example 1 by using 4′-{[4-butyl-1-(2-methoxyethyl)-6-methyl-2-oxo-1,2-dihydropyridin-3-yl]methyl}biphenyl-2-carbonitrile instead of the 4′-[(1-benzyl-4-butyl-6-methyl-2-oxo-1,2-dihydropyridin-3-yl)methyl]biphenyl-2-carbonitrile.

¹H-NMR (CDCl₃) δ:

0.88 (3H, t, J=7 Hz), 1.28-1.39 (2H, m), 1.42-1.52 (2H, m), 2.35 (3H, s), 2.43 (2H, t, J=8 Hz), 3.16 (3H, s), 3.47 (2H, brs), 3.80 (2H, s), 4.04 (2H, brs), 5.99 (1H, s), 6.89 (4H, brs), 7.29-7.46 (3H, m), 7.87 (1H, d, J=7 Hz).

### Example 10

Production of 3-{4′-{[4-butyl-1-(2-methoxyethyl)-6-methyl-2-oxo-1,2-dihydropyridin-3-yl)methyl]biphenyl-2-yl}-1,2,4-oxadiazol-5(4H)-one

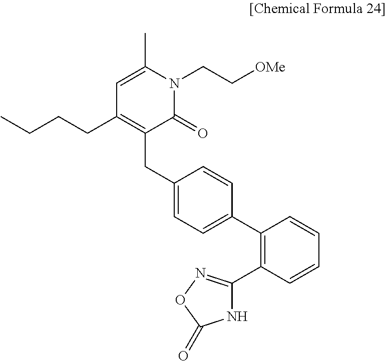

3-{4′-{[4-butyl-1-(2-methoxyethyl)-6-methyl-2-oxo-1,2-dihydropyridin-3-yl]methyl]biphenyl-2-yl}-1,2,4-oxadiazol-5(4H)-one was obtained according to the same reaction and treatment as the Processes 1 and 2 of the Example 2 by using the 4′-{[4-butyl-1-(2-methoxyethyl)-6-methyl-2-oxo-1,2-dihydropyridin-3-yl]methyl}biphenyl-2-carbonitrile obtained from the Process 3 of the Example 9 instead of the 4′-[(1-benzyl-4-butyl-6-methyl-2-oxo-1,2-dihydropyridin-3-yl)methyl]biphenyl-2-carbonitrile.

¹H-NMR (CDCl₃) δ:

0.92 (3H, t, J=7 Hz), 1.33-1.43 (2H, m), 1.46-1.56 (2H, m), 2.40 (3H, s), 2.51 (2H, t, J=8 Hz), 3.27 (3H, s), 3.57 (2H, t, J=5 Hz), 3.89 (2H, s), 4.09 (2H, t, J=5 Hz), 5.99 (1H, s), 7.15 (2H, d, J=8 Hz), 7.18 (2H, d, J=8 Hz), 7.38-7.46 (2H, m), 7.56 (1H, t, J=8 Hz), 7.76 (1H, d, J=8 Hz).

### Example 11

Production of 3-{[2′-(1H-tetrazol-5-yl)biphenyl-4-yl]methyl}-4-butyl-1-(5-ethoxypyrimidin-2-yl)-6-methylpyridin-2(1H)-one

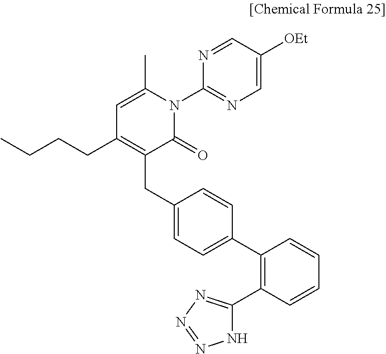

Process 1: Diketene (2.8 mL, 36 mmol) and triethylamine (2 mL, 14 mmol) were added to a toluene (30 mL) solution of 2-amino-5-ethoxypyrimidine (1.4 g, 10 mmol) and stirred for 24 hours at 85° C. The solvent was distilled off and the mixture was added ethyl acetate (10 mL) and heated under reflux for 30 min. After cooling to room temperature, the solution was filtered to obtain 3-acetyl-1-(5-ethoxypyrimidin-2-yl)-4-hydroxy-6-methylpyridin-2(1H)-one (1.10 g, 38%) as a black solid.

¹H-NMR (CDCl₃) δ:

1.51 (3H, t, J=7 Hz), 1.99 (3H, s), 2.68 (3H, s), 4.22 (2H, t, J=7 Hz), 5.93 (1H, s), 8.52 (2H, s).

Process 2:

80% aqueous solution of sulfuric acid (3 mL) was added to 3-acetyl-1-(5-ethoxypyrimidin-2-yl)-4-hydroxy-6-methylpyridin-2(1H)-one (400 mg, 1.4 mmol) and stirred for 15 minutes at 150° C. The mixture was added ice water and 5 N aqueous solution of sodium hydroxide and pH was adjusted to 8.0. The solution was then filtered. The filtrate was concentrated in vacuo and the residues obtained were subjected to silica gel column chromatography (chloroform/methanol=10:1) to obtain 1-(5-ethoxypyrimidin-2-yl)-4-hydroxy-6-methylpyridin-2(1H)-one (206 mg, 60%) as a pale yellow amorphous.

¹H-NMR (CD₃OD) δ:

1.48 (3H, t, J=7 Hz), 1.93 (3H, s), 4.29 (2H, t, J=7 Hz), 5.75 (1H, d, J=2 Hz), 6.03 (1H, d, J=2 Hz), 8.61 (2H, s).

Process 3:

Lithium chloride (41 mg, 0.97 mmol), N,N-diisopropylethylamine (338 μL, 1.9 mmol), and 4′-bromo methyl-2-cyanobiphenyl (238 mg, 0.87 mmol) were added to a N,N-dimethylformamide (5 mL) solution of 1-(5-ethoxypyrimidin-2-yl)-4-hydroxy-6-methylpyridin-2(1H)-one (240 mg, 0.97 mmol), and stirred overnight at 70° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (chloroform/methanol=10:1) to obtain 4′-{[1-(5-ethoxypyrimidin-2-yl)-4-hydroxy-6-methyl-2-oxo-1,2-dihydropyridin-3-yl]methyl}biphenyl-2-carbonitrile (63 mg, 15%) as pale yellow oil.

¹H-NMR (CDCl₃) δ:

1.43 (3H, t, J=7 Hz), 1.85 (3H, s), 3.90 (2H, s), 4.09 (2H, q, J=7 Hz), 5.91 (1H, s), 7.35-7.48 (6H, m), 7.58 (1H, dt, J=1, 8 Hz), 7.71 (1H, dd, J=1, 8 Hz), 8.42 (2H, s).

Process 4:

Pyridine (52 mg, 0.66 mmol) and trifluoromethane sulfonic anhydride (92 μL, 0.55 mmol) were added to a dichloromethane (3.0 mL) solution of 4′-{[1-(5-ethoxypyrimidin-2-yl)-4-hydroxy-6-methyl-2-oxo-1,2-dihydropyridin-3-yl]methyl}biphenyl-2-carbonitrile (80 mg, 0.18 mmol) at room temperature and stirred for 2 hours at room temperature. The reaction solution was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain a pale yellow amorphous. Under argon atmosphere, n-butyl zinc bromide (0.5 mol/L tetrahydrofuran solution, 1.2 mL, 0.58 mmol) was added to a toluene (2 mL) solution of the pale yellow amorphous obtained from the above and dichloro [1,1′-bis(diphenylphosphino)ferrocene]palladium (II) dichloromethane complex (23 mg, 0.029 mmol) at 0° C. and stirred for 1.5 hours at 90° C. The reaction mixture was added ethyl acetate and filtered through a pad of celite. The filtrate was added water to separate the organic layer, and the aqueous layer was extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain 4′-{[4-butyl-1-(5-ethoxypyrimidin-2-yl)-6-methyl-2-oxo-1,2-dihydropyridin-3-yl]methyl}biphenyl-2-carbonitrile (70 mg, 74%) as pale yellow oil.

¹H-NMR (CDCl₃) δ:

0.92 (3H, t, J=7 Hz), 1.33-1.42 (2H, m), 1.45-1.54 (5H, m), 1.97 (3H, s), 2.55 (2H, t, J=8 Hz), 4.02 (2H, s), 4.20 (2H, q, J=7 Hz), 6.00 (1H, s), 7.35-7.48 (6H, m), 7.60 (1H, dt, J=1, 8 Hz), 7.73 (1H, dd, J=1, 8 Hz), 8.51 (2H, s).

Process 5:

Trimethylsilyl azide (230 μL, 1.8 mmol) and dibutyltin oxide (4 mg, 0.018 mmol) were added to a toluene (1.5 mL) solution of 4′-{[4-butyl-1-(5-ethoxypyrimidin-2-yl)-6-methyl-2-oxo-1,2-dihydropyridin-3-yl]methyl}biphenyl-2-carbonitrile (18 mg, 0.035 mmol) and heated under reflux for 16 hours under argon atmosphere. The reaction solvent was distilled off and the residues obtained were separated and purified by silica gel column chromatography (chloroform:methanol=20:1) to obtain 3-{[2′-(1H-tetrazol-5-yl)biphenyl-4-yl]methyl}-4-butyl-1-(ethoxypyrimidin-2-yl)-6-methylpyridin-2(1H)-one (6 mg, 31%) as pale yellow oil.

¹H-NMR (CDCl₃) δ:

0.93 (3H, t, J=7 Hz), 1.35-1.60 (7H, m), 1.96 (3H, s), 2.58 (2H, t, J=8 Hz), 3.89 (2H, s), 4.16 (2H, q, J=7 Hz), 6.07 (1H, s), 7.02 (2H, d, J=8 Hz), 7.16 (2H, d, J=8 Hz), 7.34-7.57 (3H, m), 8.06 (1H, d, J=7 Hz), 8.43 (2H, s).

### Example 12

Production of 3-{4′-{[4-butyl-1-(5-ethoxypyrimidin-2-yl)-6-methyl-2-oxo-1,2-dihydropyridin-3-yl]methyl}biphenyl-2-yl}-1,2,4-oxadiazol-5(4H)-one

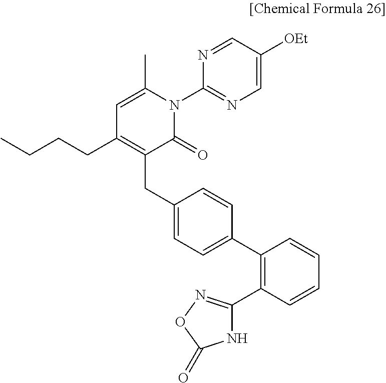

Process 1:

Sodium hydrogen carbonate (320 mg, 3.8 mmol) was added to a dimethyl sulfoxide solution (4 mL) of hydroxylamine hydrochloride (221 mg, 3.2 mmol) and stirred for 1 hour at 40° C. The dimethyl sulfoxide solution (2 mL) of the 4′-{[4-butyl-1-(5-ethoxypyrimidin-2-yl)-6-methyl-2-oxo-1,2-dihydropyridin-3-yl]methyl}biphenyl-2-carbonitrile (65 mg, 0.13 mmol) obtained in the Process 4 of the Example 11 was added to the reaction mixture and stirred for 16 hours at 90° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain pale yellow oil.

Process 2:

1,1-carbonyldiimidazole (51 mg, 0.31 mmol) and 1,8-diazabicyclo [5.4.0]undec-7-ene (47 μL, 0.31 mmol) were added to the N,N-dimethylformamide solution (1.5 mL) of the pale yellow oil obtained from the above and stirred for 2 hours at room temperature. After completion of the reaction, the reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain 3-{4′-{[4-butyl-1-(5-ethoxypyrimidin-2-yl)-6-methyl-2-oxo-1,2-dihydropyridin-3-yl]methyl}biphenyl-2-yl}-1,2,4-oxadiazol-5(4H)-one (6 mg, 18%) as pale yellow oil.

¹H-NMR (CDCl₃) δ:

0.94 (3H, t, J=7 Hz), 1.35-1.45 (2H, m), 1.48 (3H, t, J=7 Hz), 1.50-1.59 (2H, m), 1.96 (3H, s), 2.58 (2H, t, J=8 Hz), 3.94 (2H, s), 4.18 (2H, q, J=7 Hz), 6.04 (1H, s), 7.16 (2H, d, J=8 Hz), 7.27 (2H, d, J=8 Hz), 7.35-7.45 (2H, m), 7.55 (1H, t, J=8 Hz), 7.73 (1H, d, J=8 Hz), 8.47 (2H, s).

### Example 13

Production of 3-{[2′-(1H-tetrazol-5-yl)biphenyl-4-yl]methyl}-4-butyl-1-(5-methoxypyrimidin-2-yl)-6-methylpyridin-2(1H)-one

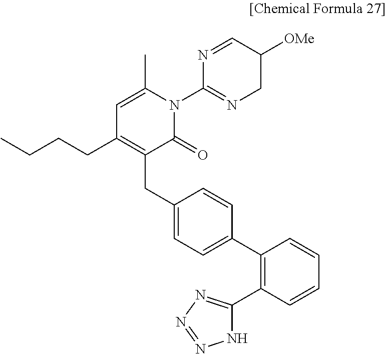

Process 1:

3-acetyl-4-hydroxy-1-(5-methoxypyrimidin-2-yl)-6-methylpyridin-2(1H)-one was obtained according to the same reaction and treatment as the Process 1 of the Example 11 by using 2-amino-5-methoxypyrimidine instead of the 2-amino-5-ethoxypyrimidine.

¹H-NMR (CDCl₃) δ:

1.99 (3H, s), 2.68 (3H, s), 4.01 (3H, s), 5.94 (1H, s), 8.55 (2H, s).

Process 2:

4-hydroxy-1-(5-methoxypyrimidin-2-yl)-6-methylpyridin-2(1H)-one was obtained according to the same reaction and treatment as the Process 2 of the Example 11 by using 3-acetyl-4-hydroxy-1-(5-methoxypyrimidin-2-yl)-6-methylpyridin-2(1H)-one instead of the 3-acetyl-1-(5-ethoxypyrimidin-2-yl)-4-hydroxy-6-methylpyridin-2(1H)-one.

¹H-NMR (CD₃OD) δ:

1.93 (3H, s), 4.03 (3H, s), 5.76 (1H, d, J=2 Hz), 6.04 (1H, d, J=2 Hz), 8.64 (2H, s).

Process 3:

4′-{[4-hydroxy-1-(5-methoxypyrimidin-2-yl)-6-methyl-2-oxo-1,2-dihydropyridin-3-yl]methyl}biphenyl-2-carbonitrile was obtained according to the same reaction and treatment as the Process 3 of the Example 11 by using 4-hydroxy-1-(5-methoxypyrimidin-2-yl)-6-methylpyridin-2(1H)-one instead of the 1-(5-ethoxypyrimidin-2-yl)-4-hydroxy-6-methylpyridin-2(1H)-one.

¹H-NMR (CDCl₃) δ:

1.83 (3H, s), 3.84 (3H, s), 3.88 (2H, s), 5.92 (1H, s), 7.34-7.48 (6H, m), 7.58 (1H, dt, J=1, 8 Hz), 7.71 (1H, d, J=8 Hz), 8.42 (2H, s).

Process 4:

4′-{[4-butyl-1-(5-methoxypyrimidin-2-yl)-6-methyl-2-oxo-1,2-dihydropyridin-3-yl]methyl}biphenyl-2-carbonitrile was obtained according to the same reaction and treatment as the Process 4 of the Example 11 by using 4′-{[4-hydroxy-1-(5-methoxypyrimidin-2-yl)-6-methyl-2-oxo-1,2-dihydropyridin-3-yl]methyl}biphenyl-2-carbonitrile instead of the 4′-{[1-(5-ethoxypyrimidin-2-yl)-4-hydroxy-6-methyl-2-oxo-1,2-dihydropyridin-3-yl]methyl}biphenyl-2-carbonitrile.

¹H-NMR (CDCl₃) δ:

0.92 (3H, t, J=7 Hz), 1.33-1.43 (2H, m), 1.44-1.54 (2H, m), 1.97 (3H, s), 2.55 (2H, t, J=8 Hz), 3.97 (3H, s), 4.02 (2H, s), 6.00 (1H, s), 7.35-7.48 (6H, m), 7.60 (1H, dt, J=1, 8 Hz), 7.73 (1H, dd, J=1, 8 Hz), 8.53 (2H, s).

Process 5:

3-{[2′-(1H-tetrazol-5-yl)biphenyl-4-yl]methyl}-4-butyl-1-(m ethoxypyrimidin-2-yl)-6-methylpyridin-2(1H)-one was obtained according to the same reaction and treatment as the Process 5 of the Example 11 by using 4′-{[4-butyl-1-(5-methoxypyrimidin-2-yl)-6-methyl-2-oxo-1,2-dihydropyridin-3-yl]methyl}biphenyl-2-carbonitrile instead of the 4′-{[4-butyl-1-(5-ethoxypyrimidin-2-yl)-6-methyl-2-oxo-1,2-dihydropyridin-3-yl]methyl}biphenyl-2-carbonitrile.

¹H-NMR (CDCl₃) δ:

0.92 (3H, t, J=7 Hz), 1.33-1.46 (2H, m), 1.49-1.59 (2H, m), 1.90 (3H, s), 2.54 (2H, t, J=8 Hz), 3.72 (3H, s), 3.84 (2H, s), 6.08 (1H, s), 6.96 (2H, d, J=8 Hz), 7.01 (2H, d, J=8 Hz), 7.31-7.46 (3H, m), 7.93 (1H, d, J=7 Hz), 8.34 (2H, s).

### Example 14

Production of 3-{4′-{[4-butyl-1-(5-methoxypyrimidin-2-yl)-6-methyl-2-oxo-1,2-dihydropyridin-3-yl] methyl}biphenyl-2-yl}-1,2,4-oxadiazol-5(4H)-one

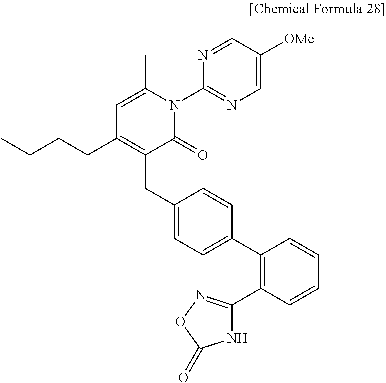

3-{4′-{[4-butyl-1-(5-methoxypyrimidin-2-yl)-6-methyl-2-oxo-1,2-dihydropyridin-3-yl]methyl}biphenyl-2-yl}-1,2,4-oxadiazol-5(4H)-one was obtained according to the same reaction and treatment as the Process 1 and 2 of the Example 12 by using the 4′-{[4-butyl-1-(5-methoxypyrimidin-2-yl)-6-methyl-2-oxo-1,2-dihydropyridin-3-yl]methyl}biphenyl-2-carbonitrile obtained in the Process 4 of the Example 13 instead of the 4′-{[4-butyl-1-(5-ethoxypyrimidin-2-yl)-6-methyl-2-oxo-1,2-dihydropyridin-3-yl]methyl}biphenyl-2-carbonitrile.

¹H-NMR (CDCl₃) δ:

0.94 (3H, t, J=7 Hz), 1.36-1.47 (2H, m), 1.51-1.61 (2H, m), 1.96 (3H, s), 2.59 (2H, t, J=8 Hz), 3.92 (2H, s), 3.95 (3H, s), 6.06 (1H, s), 7.16 (2H, d, J=8 Hz), 7.24 (2H, d, J=8 Hz), 7.35-7.44 (2H, m), 7.54 (1H, t, J=8 Hz), 7.76 (1H, d, J=8 Hz), 8.49 (2H, s).

### Example 15

Production of 3-{[2′-(1H-tetrazol-5-yl)biphenyl-4-yl]methyl}-4-butyl-6-methyl-1-(pyridin-2-yl)pyridin-2(1H)-one

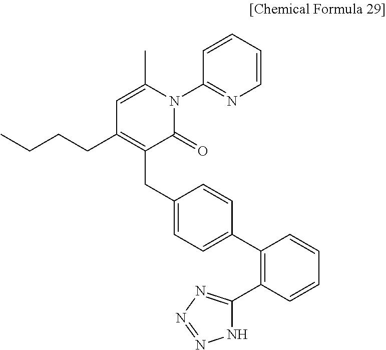

Process 1:

Sodium hydride (87 mg, 20 mmol) and 4′-bromomethyl-2-cyano biphenyl (2.7 g, 10 mmol) were added to a N,N-dimethylformamide (10 mL) solution of 4-hydroxy-6-methyl-2-pyrone (1.3 g, 10 mmol) at room temperature and stirred overnight at 70° C. The reaction solution was added water and extracted with chloroform. The organic layer was combined, washed with water and brine, dried over anhydrous sodium sulfate, and concentrated in vacuo. The residues obtained were subjected to silica gel column chromatography (chloroform/methanol=10:1) to obtain 4′-[(4-hydroxy-6-methyl-2-oxo-2H-pyran-3-yl)methyl]biphenyl-2-carbonitrile (550 mg, 17%) as pale yellow oil.

¹H-NMR (CD₃OD) δ:

2.18 (3H, s), 3.76 (2H, s), 5.97 (1H, s), 7.38-7.56 (6H, m),

7.69 (1H, dt, J=1, 8 Hz), 7.78 (1H, d, J=8 Hz).

Process 2:

Pyridine (45 mg, 0.57 mmol) and trifluoromethane sulfonic anhydride (87 μL, 0.52 mmol) were added to a dichloromethane (1.5 mL) solution of 4′-[(4-hydroxy-6-methyl-2-oxo-2H-pyran-3-yl)methyl]biphenyl-2-carbonitrile (55 mg, 0.17 mmol) at room temperature and stirred for 2 hours at room temperature. The reaction solution was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain pale yellow oil. Under argon atmosphere, n-butyl zinc bromide (0.5 mol/L tetrahydrofuran solution, 0.8 mL, 0.40 mmol) was added to a toluene (1 mL) solution of the pale yellow oil obtained from the above and dichloro[1,1′-bis(diphenylphosphino)ferrocene] palladium (II) dichloromethane complex (22 mg, 0.027 mmol) at 0° C. and stirred for 1.5 hours at 90° C. The reaction mixture was added ethyl acetate and filtered through a pad of celite. The filtrate was added water to separate the organic layer, and the aqueous layer was extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain 4′-[(4-butyl-6-methyl-2-oxo-2H-pyran-3-yl)methyl]biphenyl-2-carbonitrile (28 mg, 74%) as pale yellow oil.

¹H-NMR (CDCl₃) δ:

0.90 (3H, t, J=7 Hz), 1.31-1.51 (4H, m), 2.23 (3H, s), 2.46 (2H, t, J=8 Hz), 3.92 (2H, s), 5.90 (1H, s), 7.35 (2H, d, J=8 Hz), 7.38-7.51 (4H, m), 7.62 (1H, dt, J=1, 8 Hz), 7.74 (1H, dd, J=1, 8 Hz).

Process 3:

Under argon atmosphere, trimethylaluminum (2 mol/L heptane solution, 0.22 mL, 0.44 mmol) was added to a 1,2-dichloroethane (3 mL) solution of 2-aminopyridine (25 mg, 0.27 mmol) at room temperature and stirred for 1 hour at the same temperature. After that, 1,2-dichloroethane solution (2 mL) of 4′-[(4-butyl-6-methyl-2-oxo-2H-pyran-3-yl)methyl]biphenyl-2-carbonitrile (32 mg, 0.090 mmol) was added dropwise thereto at room temperature and heated under reflux for 16 hours. Aqueous solution of ammonium chloride and chloroform were added to the reaction mixture, which was then filtered through a pad of celite. The organic layer was removed from the filtrate and the aqueous layer was extracted with chloroform. The organic layer was combined, washed with water and brine, dried over anhydrous sodium sulfate, and concentrated in vacuo. The residues obtained were subjected to silica gel column chromatography (hexane/acetone=1:1) to obtain 4′-{{4-butyl-6-methyl-2-oxo-1-(pyridin-2-yl)-1,2-dihydropyridin-3-yl}methyl}biphenyl-2-carbonitrile (17 mg, 47%) as pale yellow oil.

¹H-NMR (CDCl₃) δ:

0.93 (3H, t, J=7.2 Hz), 1.34-1.44 (2H, m), 1.46-1.56 (2H, m), 1.96 (3H, s), 2.56 (2H, t, J=7.8 Hz), 4.02 (2H, s), 6.03 (1H, s), 7.35-7.48 (8H, m), 7.60 (1H, dt, J=1.2, 7.7 Hz), 7.73 (1H, dd, J=1.2, 7.7 Hz), 7.88 (1H, dt, J=1.8, 7.7 Hz), 8.65 (1H, dd, J=1.8, 5.5 Hz).

Process 4:

Under argon atmosphere, trimethylsilyl azide (211 μL, 1.61 mmol) and dibutyltin oxide (4.0 mg, 0.016 mmol) were added to a toluene (1.5 mL) solution of 4′-{{4-butyl-6-methyl-2-oxo-1-(pyridin-2-yl)-1,2-dihydropyridin-3-yl}methyl}biphenyl-2-carbonitrile (14 mg, 0.032 mmol) and heated under reflux for 16 hours. The reaction solution was distilled off and the residues obtained were separated and purified by silica gel column chromatography (chloroform:methanol=20:1) to obtain

3-{[2′-(1H-tetrazol-5-yl)biphenyl-4-yl]methyl}-4-butyl-6-methyl-1-(pyridin-2-yl)pyridin-2(1H)-one (11 mg, 71%) as a pale yellow amorphous.

¹H-NMR (CDCl₃) δ:

0.91 (3H, t, J=7.2 Hz), 1.31-1.42 (2H, m),

1.47-1.57 (2H, m), 1.89 (3H, s), 2.50 (2H, t, J=7.8 Hz),

3.77 (2H, s), 6.10 (1H, s), 6.86 (4H, brs),

7.05-7.11 (1H, m), 7.16 (1H, d, J=7.8 Hz),

7.28-7.44 (3H, m), 7.57-7.68 (1H, m), 7.84 (1H, d, J=7.3 Hz),

7.84 (1H, d, J=7.3 Hz), 8.36 (1H, d, J=3.9 Hz).

### Example 16

Production of 3-{4′-{[4-butyl-6-methyl-2-oxo-1-(pyridin-2-yl)-1,2-dihydro pyridin-3-yl]methyl}biphenyl-2-yl}-1,2,4-oxadiazol-5(4H)-one

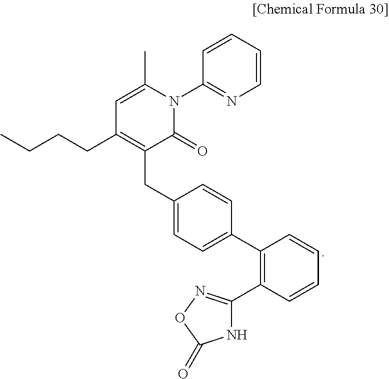

Process 1:

Sodium hydrogen carbonate (378 mg, 4.5 mmol) was added to a dimethyl sulfoxide solution (3 mL) of hydroxylamine hydrochloride (260 mg, 3.8 mmol) and stirred for 1 hour at 40° C. The reaction mixture was added the dimethyl sulfoxide solution (2 mL) of 4′-{{4-butyl-6-methyl-2-oxo-1-(pyridin-2-yl)-1,2-dihydropyridin-3-yl}methyl}biphenyl-2-carbonitrile (65 mg, 0.15 mmol) obtained in the Process 3 of the Example 15 and stirred for 16 hours at 90° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain pale yellow oil.

Process 2: 1,1′-carbonyldiimidazole (61 mg, 0.38 mmol) and 1,8-diazabicyclo [5.4.0]undec-7-ene (56 μL, 0.38 mmol) were added to the N,N-dimethylformamide solution (2 mL) of the pale yellow oil obtained from the above and stirred for 2 hours at room temperature. After completion of the reaction, the reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain 3-{4′-{[4-butyl-6-methyl-2-oxo-1-(pyridin-2-yl)-1,2-dihydro pyridin-3-yl]methyl}biphenyl-2-yl}-1,2,4-oxadiazol-5(4H)-one (18 mg, 26%) as pale yellow oil.

¹H-NMR (CDCl₃) δ:

0.96 (3H, t, J=7.2 Hz), 1.38-1.50 (2H, m),

1.55-1.65 (2H, m), 1.96 (3H, s), 2.62 (2H, t, J=7.8 Hz),

3.93 (2H, s), 6.12 (1H, s), 7.19 (2H, d, J=8.0 Hz),

7.24 (2H, d, J=8.0 Hz), 7.32-7.58 (5H, m),

7.79 (1H, d, J=7.6 Hz), 7.88 (1H, t, J=7.6 Hz),

8.59 (1H, d, J=3.9 Hz).

### Example 17

Production of 3-{[2′-(1H-tetrazol-5-yl)biphenyl-4-yl]methyl}-1-benzyl-6-m ethyl-4-propoxypyridin-2(1H)-one

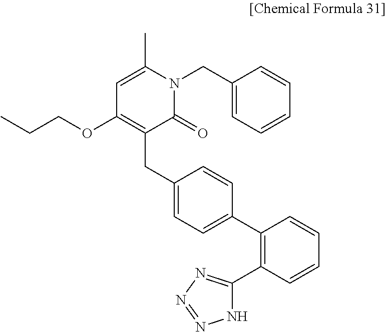

Process 1:

Sodium hydride (34 mg, 0.8 mmol) and 1-iodopropane (75 μL, 0.8 mmol) were added to a N,N-dimethylformamide (3 mL) solution of the 4′-[(1-benzyl-4-hydroxy-6-methyl-2-oxo-1,2-dihydropyridin-3-yl)methyl]biphenyl-2-carbonitrile (105 mg, 0.3 mmol) obtained in the Process 2 of the Example 1 at room temperature and stirred overnight at room temperature. The reaction solution was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=2:1) to obtain 4′-[(1-benzyl-6-methyl-2-oxo-4-propoxy-1,2-dihydropyridin-3-yl)methyl]biphenyl-2-carbonitrile (109 mg, 38%) as pale yellow oil.

¹H-NMR (CDCl₃) δ:

1.03 (3H, t, J=7 Hz), 1.73-1.88 (2H, m), 2.26 (3H, s), 3.98 (2H, t, J=6 Hz), 4.00 (2H, s), 5.35 (2H, s), 5.93 (1H, s), 7.09-7.54 (11H, m), 7.59 (1H, dt, J=1, 8 Hz), 7.73 (1H, dt, J=1, 8 Hz).

Process 2:

Under argon atmosphere, trimethylsilyl azide (730 μL, 5.6 mmol) and dibutyltin oxide (14 mg, 0.056 mmol) were added to a toluene (3 mL) solution of 4′-[(1-benzyl-6-methyl-2-oxo-4-propoxy-1,2-dihydropyridin-3-yl)methyl]biphenyl-2-carbonitrile (50 mg, 0.11 mmol) and heated under reflux for 16 hours. The reaction solvent was distilled off and the residues obtained were separated and purified by silica gel column chromatography (chloroform:methanol=20:1) to obtain 3-{[2′-(1H-tetrazol-5-yl)biphenyl-4-yl]methyl}-1-benzyl-6-methyl-4-propoxypyridin-2(1H)-one (27 mg, 50%) as pale yellow oil.

¹H-NMR (CDCl₃) δ:

0.99 (3H, t, J=7 Hz), 1.74-1.85 (2H, m), 2.24 (3H, s),

3.81 (2H, s), 3.97 (2H, t, J=6 Hz), 5.17 (2H, s),

5.98 (1H, s), 6.90 (2H, d, J=8 Hz), 6.99 (2H, d, J=7 Hz),

7.09 (2H, d, J=8 Hz), 7.17-7.52 (6H, m),

7.82 (1H, d, J=8 Hz).

### Example 18

Production of 3-{4′-[(1-benzyl-6-methyl-2-oxo-4-propoxy-1,2-dihydropyridin-3-yl)methyl]biphenyl-2-yl}-1,2,4-oxadiazol-5(4H)-one

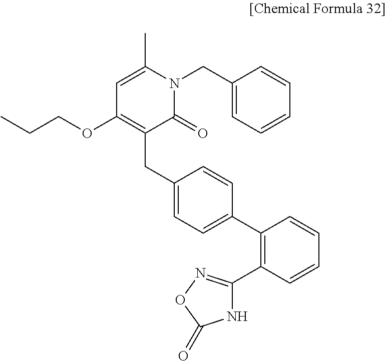

Process 1:

Sodium hydrogen carbonate (427 mg, 5.1 mmol) was added to a dimethyl sulfoxide solution (3 mL) of hydroxylamine hydrochloride (294 mg, 4.2 mmol) and stirred for 1 hour at 40° C. The reaction mixture was added the dimethyl sulfoxide solution (2 mL) of 4′-[(1-benzyl-6-methyl-2-oxo-4-propoxy-1,2-dihydropyridin-3-yl)methyl]biphenyl-2-carbonitrile (76 mg, 0.17 mmol) obtained in the Process 1 of the Example 17, and stirred for 16 hours at 90° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain pale yellow oil.

Process 2:

1,1′-carbonyldiimidazole (69 mg, 0.42 mmol) and 1,8-diazabicyclo [5.4.0]undec-7-ene (63 mg, 0.42 mmol) were added to the N,N-dimethylformamide solution (2 mL) of the pale yellow oil obtained from the above, and stirred for 2 hours at room temperature. After completion of the reaction, the reaction solution was added water and extracted with ethyl acetate. The organic layer was combined, washed with water and brine, dried over anhydrous sodium sulfate, and concentrated in vacuo. The residues obtained were subjected to silica gel column chromatography (ethyl acetate) to obtain 3-{4′-[(1-benzyl-6-methyl-2-oxo-4-propoxy-1,2-dihydropyridin-3-yl)methyl]biphenyl-2-yl}-1,2,4-oxadiazol-5(4H)-one (40 mg, 51%) as pale yellow oil.

¹H-NMR (CDCl₃) δ:

1.04 (3H, t, J=7 Hz), 1.78-1.88 (2H, m), 2.24 (3H, s),

3.87 (2H, s), 3.99 (2H, t, J=6 Hz), 5.19 (2H, s),

5.96 (1H, s), 7.02 (2H, d, J=7 Hz), 7.17 (2H, d, J=8 Hz),

7.14-7.57 (8H, m), 7.67 (1H, d, J=8 Hz).

### Test Example 1

### Angiotensin II Antagonistic Activity in Isolated Rabbit Blood Vessels

By using a specimen of isolated rabbit blood vessels, antagonistic activity of the compounds of the invention against angiotensin II type 1 receptor was estimated from a dose-response curve of angiotensin II-induced blood vessel contraction. Specifically, the specimen of thoracic aorta ring of a rabbit (New Zealand White: male, 2.4 to 3.0 kg) was suspended in a magnus bath filled with Krebs-Henseleite buffer (composition: 118 mM NaCl, 4.7 mM KCl, 2.55 mM CaCl₂, 1.18 mM MgSO₄, 1.18 mM KH₂PO₄, 24.88 mM NaHCO₃, and 11.1 mM D-glucose), and angiotensin II (10 nM)-induced contraction was obtained in the presence of the compounds of each example (1 nmol/L to 10 μmol/L). During the measurement, the inside temperature of the magnus bath was maintained at 37° C. and the bath was continuously ventilated with a sufficient amount of mixed gas (95% O₂ and 5% CO₂). The angiotensin II-induced contraction was converted into a relative value (%) that is based on the angiotensin II (10 nM)-induced contraction in the absence of the compounds of each example. From the concentration-response curve obtained therefrom, 50% inhibition concentration (IC₅₀ value) was calculated by using SAS Preclinical Package Ver5.0 (trade name, manufactured by SAS institute Japan Co., Tokyo, Japan), which is a statistical analysis program.

As a result, it was found that the compound described in the example has an angiotensin II inhibition activity at 10 μM concentration. Inhibitory activity of the compounds (i.e., IC₅₀ value) is given in the Table 1. As shown in the Table 1, it was confirmed that the compounds of the invention have a potent angiotensin II antagonistic activity.

TABLE 1

| Example No. | IC₅₀ (μM) |
|---|---|
| 7 | 0.58 |
| 8 | 0.39 |
| 9 | 0.16 |
| 10 | 0.19 |
| 11 | 0.33 |
| 12 | 0.46 |
| 14 | 0.54 |
| 15 | 0.46 |

### Test Example 2

### PPARγ Activation Activity

The agonistic activity of the compounds of the invention on PPARγ was measured based on the transfection assay using COST cells (DS Pharma Biomedical Co., Ltd., Osaka, Japan), which are the cell line derived from the kidney of the African green monkey. COST cells were cultured under 5% CO₂ concentration, and DMEM medium containing 10% fetal bovine serum, glutamic acid, and antibiotics was used as a medium.

As an expression vector, a chimera in which DNA binding domain of Ga14, which is a yeast transcription factor, and ligand binding domain of human PPARγ2 are fused, i.e., a fused product between the amino acids 1 to 147 of Gal4 transcription factor and the amino acids 182 to 505 of human PPARγ2, was used. Furthermore, as a reporter vector, a firefly luciferase containing five copies of Gal4 recognition sequence in the promoter region was used. Plasmid transfection to the cells was performed according to a method which uses jetPEI (trade name, manufactured by Funakoshi Co., Ltd., Tokyo, Japan). Furthermore, β-galactosidase expression vector was employed as an internal standard.

After the transfection of the cells, the medium was replaced with a DMEM medium (containing 1% serum) added with the test compound, and the cells were further cultured for 16 hours. After that, the luciferase activity and β-galactosidase activity in the cell lysis solution were measured.

For the present test, dimethyl sulfoxide (DMSO) was used for dissolution and dilution of the test compounds, and during the cell treatment, the DMSO concentration in DMEM medium (containing 1% serum) was adjusted to 0.1%. The 50% effective concentration of the test compound (EC₅₀, 50% effect concentration) was calculated by using SAS Preclinical Package Ver 5.0 (trade name, manufactured by SAS institute Japan Co., Tokyo, Japan), which is a statistical analysis program.

The results are given in the Table 2. As shown in the Table 2, it was confirmed that the compounds of the present invention have a potent PPARγ activation activity. Under the same condition, the PPARγ activation activity of telmisartan, i.e., EC₅₀, was 1 to 5 μM.

TABLE 2

| Example No. | EC₅₀ (μM) |
|---|---|
| 1 | 0.40 |
| 2 | 0.26 |
| 3 | 0.32 |
| 4 | 0.20 |
| 5 | 0.50 |
| 6 | 0.26 |
| 7 | 0.27 |
| 8 | 0.20 |
| 9 | 0.56 |
| 10 | 0.45 |
| 11 | 2.22 |
| 12 | 0.59 |
| 14 | 1.27 |
| 15 | 1.66 |
| 16 | 1.40 |
| 17 | 0.58 |
| 18 | 0.14 |

From the results obtained above, it was confirmed that the compounds represented by the general formula (1) of the present invention have both a potent angiotensin II receptor antagonistic activity and a PPARγ activation activity. Thus, it was found that the compounds (I) represented by the formula (1) of the present invention and pharmaceutically acceptable salts thereof are useful as an effective component of a prophylactic and/or therapeutic agent for disorders involved with angiotensin II and PPARγ, for example, hypertension, heart diseases, angina pectoris, cerebrovascular disorders, cerebral circulatory disorders, ischemic peripheral circulatory disorders, renal diseases, arteriosclerosis, inflammatory diseases, type 2 diabetes, diabetic complications, insulin resistance syndrome, syndrome X, metabolic syndrome, and hyperinsulinemia.

### INDUSTRIAL APPLICABILITY

The invention provides a novel compound of 2-pyridone derivative represented by the formula (1) or a salt thereof, or a solvate thereof, which has both an angiotensin II receptor antagonistic activity and a PPARγ activation activity. They can be used as an effective component of a novel pharmaceutical product, i.e., a prophylactic and/or therapeutic agent for disorders that are related with angiotensin II and PPARγ, for example, hypertension, heart diseases, angina pectoris, cerebrovascular disorders, cerebral circulatory disorders, ischemic peripheral circulatory disorders, renal diseases, arteriosclerosis, inflammatory diseases, type 2 diabetes, diabetic complications, insulin resistance syndrome, syndrome X, metabolic syndrome, and hyperinsulinemia, and therefore have an industrial applicability.

1. A compound represented by the formula (1) below: 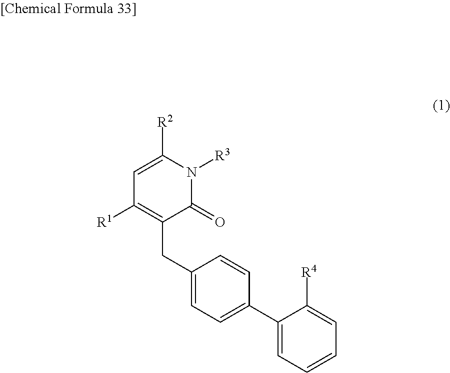
[in the formula, R¹ represents a C₁₋₆ alkyl group or a C₁₋₆ alkoxy group,
R² represents a C₁₋₆ alkyl group or a C₃₋₈ cyclolalkyl group,
R³ represents a C₁₋₆ alkyl group, a C₆₋₁₀ aryl-C₁₋₆ alkyl group, a C₁₋₆ alkoxy-C₁₋₆ alkyl group, or the following formula (2): 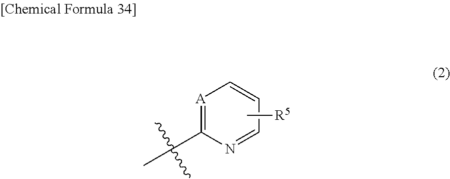
(in which A represents a nitrogen atom or CH and R⁵ represents a hydrogen atom or a C₁₋₆ alkoxy group), and
R⁴ represents the following formula (3) or the formula (4): 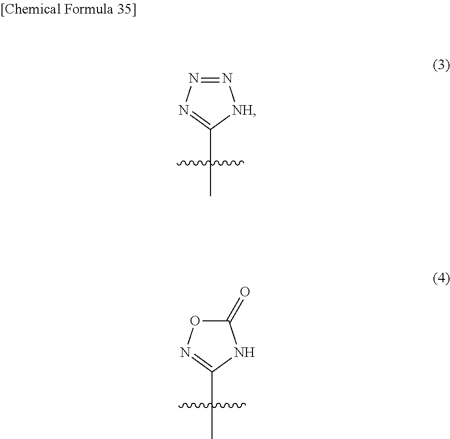
or a salt thereof, or a solvate thereof].

2. The compound according to claim 1 or the salt thereof, or the solvate thereof, in which the compound represented by the formula (1) is a compound selected from a group consisting of:
3-{[2′-(1H-tetrazol-5-yl)biphenyl-4-yl]methyl}-1-benzyl-4-butyl-6-methylpyridin-2(1H)-one,
3-{4′-[(1-benzyl-4-butyl-6-methyl-2-oxo-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-1-benzyl-6-methyl-4-propylpyridin-2 (1H)-one,
3-{4′-[(1-benzyl-6-methyl-2-oxo-4-propyl-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-1-benzyl-6-methyl-4-pentylpyridin-2(1H)-one,
3-{4′-[(1-benzyl-6-methyl-2-oxo-4-pentyl-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-4-butyl-6-methyl-1-phenethylpyridin-2(1H)-one,
3-{4′-[(4-butyl-6-methyl-2-oxo-1-phenethyl-1,2-dihydropyridin-3-yl)methyl]biphenyl-2-yl}-1,2,4-oxadiazol-5(4H)-one,
3-{[2′-(1H-tetrazol-5-yl)biphenyl-4-yl]methyl}-4-butyl-1-(2-methoxyethyl)-6-methylpyridin-2(1H)-one,
3-{4′-{[4-butyl-1-(2-methoxyethyl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-1-(5-ethoxypyrimidin-2-yl)-6-methylpyridin-2(1H)-one,
3-{4′-{[4-butyl-1-(5-ethoxypyrimidin-2-yl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-1-(5-methoxypyrimidin-2-yl)-6-methylpyridin-2(1H)-one,
3-{4′-{[4-butyl-1-(5-methoxypyrimidin-2-yl)-6-methyl-2-oxo-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-4-butyl-6-methyl-1-(pyridin-2-yl)pyridin-2(1H)-one,
3-{4′-{[4-butyl-6-methyl-2-oxo-1-(pyridin-2-yl)-1,2-dihydropyridin-3-yl]methyl}biphenyl-2-yl}-1,2,4-oxadiazol-5(4H)-one,
3-{[2′-(1H-tetrazol-5-yl)biphenyl-4-yl]methyl}-1-benzyl-6-methyl-4-propoxy pyridin-2(1H)-one, and
3-{4′-[(1-benzyl-6-methyl-2-oxo-4-propoxy-1,2-dihydropyridin-3-yl)methyl]biphenyl-2-yl}-1,2,4-oxadiazol-5(4H)-one.

3. A pharmaceutical composition comprising the compound described in claim 1, or salt thereof, or solvate thereof, and a pharmaceutically acceptable carrier.

4. A pharmaceutical composition which has both angiotensin II receptor antagonistic activity and PPARγ activation activity and comprises as an effective component the compound described in claim 1 or salt thereof, or solvate thereof.

5. An agent for preventing and/or treating a circulatory disorder which comprises as an effective component the compound described in claim 1 or salt thereof, or solvate thereof.

6. The agent for preventing and/or treating a circulatory disorder according to claim 5, wherein the circulatory disorder is hypertension, heart diseases, angina pectoris, cerebrovascular disorders, cerebral circulatory disorders, ischemic peripheral circulatory disorders, renal diseases, or arteriosclerosis.

7. An agent for preventing and/or treating a metabolic disorder which comprises as an effective component the compound described in claim 1 or salt thereof, or solvate thereof.

8. The agent for preventing and/or treating a metabolic disorder according to claim 7, wherein the metabolic disorder is type 2 diabetes, diabetic retinopathy, diabetic neuropathy, diabetic nephropathy, insulin resistance syndrome, metabolic syndrome, or hyperinsulinemia.

9. A method of preventing and/or treating a circulatory disorder, characterized in that an effective amount of the compound described in claim 1 or salt thereof, or solvate thereof is administered to a patient who is in need of treatment.

10. A method of preventing and/or treating a metabolic disorder, characterized in that an effective amount of the compound described in claim 1 or salt thereof, or solvate thereof is administered to a patient who is in need of treatment.

11. Use of the compound described in claim 1 or salt thereof, or solvate thereof for producing a preparation for preventing and/or treating a circulatory disorder.

12. Use of the compound described in claim 1 or salt thereof, or solvate thereof for producing a preparation for preventing and/or treating a metabolic disorder.

13. The compound described in claim 1 or salt thereof, or solvate thereof as a prophylactic and/or therapeutic agent that has both angiotensin II receptor antagonistic activity and PPARγ activation activity.
